(19) United States
(12) Patent Application Publication
(10) Pub. No.: US 2026/0283997 A1
(43) Pub. Date: Sep. 24, 2026

(54) METHODS FOR INHIBITING RESISTANCE TO IMMUNE BLOCKADE INHIBITOR THERAPY

(71) Applicant: H. LEE MOFFITT CANCER CENTER AND RESEARCH INSTITUTE, INC., Tampa, FL (US)

(72) Inventor: Joseph MARKOWITZ, Tampa, FL (US)

(21) Appl. No.: 19/476,385

(22) PCT Filed: Apr. 22, 2024
(86) PCT No.: PCT/US2024/025735

§ 371 Date: Oct. 17, 2025

Related U.S. Application Data
(60) Provisional application No. 63/497,563, filed on Apr. 21, 2023.

(51) Int. Cl. A61K 31/24; A61K 31/223; A61K 39/00; A61K 39/395; A61P 35/00; G01N 33/5751

(52) U.S. Cl. CPC A61K 31/24; A61K 31/223; A61K 39/3955; A61P 35/00; G01N 33/5751; A61K 2039/505

(57) ABSTRACT

Disclosed are compositions and methods related to the use of nitric oxide inhibitors to reduce resistance to immune checkpoint inhibitors. In one aspect, disclosed herein are methods of detecting elevated NO or nitration of proteins in a biological sample from a subject with a cancer, and administering an NO inhibitor to the subject where immune suppressive cells have elevated levels of NO or protein nitration.

### CROSS-REFERENCE TO RELATED APPLICATIONS

This PCT application claims priority to, and the benefit of, U.S. Provisional Patent Application No. 63/497,563, filed Apr. 21, 2023, which is incorporated by reference herein in its entirety.

### STATEMENT REGARDING FEDERALLY SPONSORED RESEARCH

This invention was made with government support under Grant No. P50CA168536 and K08CA252164 awarded by the National Institutes of Health/National Cancer Institute. The government has certain rights in the invention.

### BACKGROUND

The immune system has an expanding role in fighting the development and progression of melanoma while also being harnessed to produce effective treatment strategies. Recent studies suggest that the antitumor effects of cytotoxic chemotherapy may be mediated at least in part by altering intratumoral immune infiltrates. Blockade of initiators of T cell inhibitory pathways, such as cytotoxic T-lymphocyte-associated antigen 4 (CTLA-4) and programmed cell death protein 1 (PD-1), with neutralizing monoclonal antibodies has led to remarkable regression of disease. Monoclonal antibodies against CTLA-4, ipilimumab, and PD-1, pembrolizumab and nivolumab, have been approved for patients with unresectable melanoma or after surgery for advanced melanoma. The response rates for ipilimumab and nivolumab among metastatic melanoma patients are 11% to 22% and 31% to 44%, respectively, in the metastatic setting. The response rate of single-agent anti-PD-1 is approximately 40% in the modern era, and in combination with anti-CTLA-4 therapy, response rates increase to 50% to 60% at the cost of significantly increased toxicity. In addition, interferon alpha is also approved for use by melanoma patients after surgery, although it is used less frequently now due to the success of immune checkpoint blockade. Interferon has an unfavorable toxicity profile, but is likely to benefit a select group of patients.

Both types of checkpoint blockade (targeting PD-1 and CTLA-4) are now FDA-approved for patients who have undergone surgery for metastatic melanoma, although it is unclear which patients benefit most from each adjuvant therapy. An adjuvant study comparing anti-CTLA-4 (3 mg/kg or 10 mg/kg) to interferon alfa demonstrated increased overall survival (OS) benefit for the 3 mg/kg ipilimumab group vs interferon alfa (hazard ratio, 0.78 [95% CI, 0.61, 0.99]). In the adjuvant setting, anti-PD-1 has increased relapse-free survival (RFS) benefit over anti-CTLA-4 but no OS benefit. Given these results, most centers use adjuvant anti-PD-1 therapy.

Elucidation of the immune response is critical to the development of these clinical approaches. Melanoma cells are recognized by the immune system, but the anti-tumor activity of T cells is abrogated by several mechanisms, including depletion of nutrients from the tumor microenvironment, production of reactive oxygen and nitrogen species, secretion of immune-suppressive cytokines, and induction of inhibitory immune cells. Presentation of antigens to T cells by dendritic cells (DCs) is defective in the setting of melanoma. Stimulation of DCs with type I interferons (IFN-α and IFN-β) and downstream signal transduction via the Janus kinase-signal transducer and activator of transcription (Jak-STAT) pathway are critically important to immune surveillance and the generation of effective host T-cell immune responses to cancer. Furthermore, in DCs, IFN-signaling is responsible for upregulation of class I and class II major histocompatibility complex (MHC) molecules for the presentation of antigens on DCs. The anti-tumor effects of IFN-α are dependent on STAT1 signal transduction in immune cells via phosphorylation of tyrosine 701, and Jak-STAT signaling was markedly inhibited in human peripheral blood immune cells from tumor-bearing patients.

What are needed are new ways to detect resistance to immune checkpoint inhibitors and methods of reversing this resistance leading to improved antitumor immunity.

### SUMMARY

Disclosed are methods and compositions related to the use of nitic oxide inhibitors to reduce, inhibit, or prevent resistance to immune checkpoint inhibitors.

In one aspect, disclosed herein are methods of reducing immune checkpoint inhibitor resistance comprising administering a nitric oxide inhibitor (such as, for example, nitic oxide donating compounds (for example nitroaspirin (e.g NCX-4016), NOSH-Aspirin), L-nitroarginine methyl ester (L-NAME), Aminoguanidine hydrochloride, S-Isopropylisothiourea hydrobromide, or (S)-Methylisothiourea sulfate) to a subject being treated for a disease (such as, for example, a cancer, including, but not limited to melanoma) with an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

Also disclosed herein are methods of treating, reducing, inhibiting, decreasing, ameliorating, and/or preventing a disease (such as, for example, a cancer and/or metastasis including, but not limited to melanoma) in a subject comprising administering to the subject a nitric oxide inhibitor (such as, for example, nitic oxide donating compounds (for example nitroaspirin (e.g NCX-4016), NOSH-Aspirin), L-nitroarginine methyl ester (L-NAME), Aminoguanidine hydrochloride, S-Isopropylisothiourea hydrobromide, or (S)-Methylisothiourea sulfate) and an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

In one aspect, disclosed herein are methods of reducing immune checkpoint inhibitor resistance of any preceding aspect and methods of treating, reducing, inhibiting, decreasing, ameliorating, and/or preventing a disease of any preceding aspect, wherein the nitric oxide inhibitor is administered no greater than 3 days after administration of the immune checkpoint inhibitor. In one aspect the nitric oxide inhibitor is administered at least 1, 2, 3, 4, 5 6, 7, 8, 9 10, 11, 12, 13, 14, 15, 16, 17, 18, 19, 20, 21, 22, 23, 24, 25, 26, 27, 28, 29, 30, 31, 60, 90 days prior to administration of or 5, 10, 15, 20, 25, 30, 35, 40, 45, 50, 55, 60, 75, 90, 105, 120, 150, 180 min, or 4, 5, 6, 7, 8, 9, 10, 11, 12, 13, 14, 15, 16, 17, 18, 19, 20, 21, 22, 23, 24, 36, 48, 60, or 72 hours after administration of the immune checkpoint inhibitor.

Also disclosed herein are combinations therapy comprising a nitric oxide inhibitor (such as, for example, nitic oxide donating compounds (for example nitroaspirin (e.g NCX-4016), NOSH-Aspirin), L-nitroarginine methyl ester (L-NAME), Aminoguanidine hydrochloride, S-Isopropylisothiourea hydrobromide, or (S)-Methylisothiourea sulfate) and an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

In one aspect, disclosed herein are methods of detecting resistance of a disease or condition (such as for example, cancer or immune checkpoint inhibitor toxicity) in a subject to an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) comprising a) obtaining a tissue sample from a subject that is receiving or that is likely to receive an immune checkpoint inhibitor for treatment of a disease or condition; b) measuring the level of nitration in the tissue sample (such as, for example, nitration measured by one or more of mass spectroscopy, flow cytometry, immunohistochemical staining, immunoblot, Multi-Dimensional Phenotyping Analysis Tool in R (MPATR) clustering, and/or proteomics); wherein an increase in nitration relative to a control indicates that the disease or condition is resistant to an immune checkpoint inhibitor; and c) when a subject has a disease or condition that is resistant to an immune checkpoint inhibitor, administering to the subject a nitric oxide inhibitor (such as, for example, nitic oxide donating compounds (for example nitroaspirin (e.g NCX-4016), NOSH-Aspirin), L-nitroarginine methyl ester (L-NAME), Aminoguanidine hydrochloride, S-Isopropylisothiourea hydrobromide, or (S)-Methylisothiourea sulfate).

Also disclosed herein are methods of improving, or increasing progression free survival (PFS) of a cancer (including, but not limited to melanoma) subject with a cancer resistant to conventional cancer therapy (including, but not limited to subjects with cancers that are resistant to an immune checkpoint inhibitor therapy (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), Interferon-alpha, Interferon-gamma)), the method comprising: obtaining a biological sample (such as, for example, peripheral blood mononuclear cells (PBMC)) from the subject, performing a multi-parameter phenotyping tool in R (MPATR) assay on the biological sample, analyzing the nitric oxide (NO) levels in the biological sample, measuring the immune cells subtype producing NO (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)), and administering a NO inhibitor (such as, for example, nitic oxide donating compounds (for example nitroaspirin (e.g NCX-4016), NOSH-Aspirin), L-nitroarginine methyl ester (L-NAME), L-NMMA, Aminoguanidine hydrochloride, S-Isopropylisothiourea hydrobromide, or (S)-Methylisothiourea sulfate) in combination with an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma to subjects with a cancer resistant to conventional cancer therapy having increased levels of NO in immune suppressive cells. In some aspects, the NO inhibitor is administered on day 2 and administration of immune checkpoint inhibitor therapy on day 0.

In one aspect, disclosed herein are multi-parameter phenotyping tool in R (MPATR) assay to identify nitration of proteins in an immune cell subtype, comprising: a) obtaining biological sample (such as, for example, peripheral blood mononuclear cells (PBMC)) from a subject with a cancer (such as, for example, melanoma) that is resistant to conventional cancer therapy; b) measuring the immune cell subtype (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)) producing NO; wherein the immune cell subtype are immune suppressive cells or immune effector cells; and c) measuring the nitration of proteins in the immune cells (including, but not limited to measuring nitration using mass spectrometry); wherein an increased level of nitration of proteins in immune suppressive cells is an indication that the subject needs a NO inhibitor (such as, for example, nitic oxide donating compounds (for example nitroaspirin (e.g NCX-4016), NOSH-Aspirin), L-nitroarginine methyl ester (L-NAME), L-NMMA, Aminoguanidine hydrochloride, S-Isopropylisothiourea hydrobromide, or (S)-Methylisothiourea sulfate) to overcome resistance to conventional cancer therapy (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, HIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma.

Also disclosed herein are MPATR assays of any preceding aspect, wherein the subtype of the immune cells is measured using flow cytometry panels consisting of a myeloid cell panel, a lymphoid cell panel, and a secondary panel; wherein the panels were stained with myeloid antibodies (such as for example, antibodies that bind DAF-FM, HLA-DR-PE-Cy7, CD33-APC, CD11b-BV421, CD14-BUV395, CD15-BV510, or CD11c-PE), or lymphoid antibodies (such as, for example, antibodies that bind DAF-FM, CD3-BUV395, CD8-BV510, CD11c-PE, CD56-BV421, CD4-AF700, CD19-PE-Dazzle, CD25-PE-Cy7, or CD127-APC). In some aspects, the panels can further comprise a secondary panel of antibodies, including, but not limited to antibodies that bind IFNy, PD-L1, CTLA4, Arginase 1, FoxP3, TCR-ζ, or CD69.

In one aspect, disclosed herein are MPATR assays of any preceding aspect, wherein the proteins identified for nitration comprises STAT1, Actin, NF-kB, STAT3, PUS7, FAK1, or HDAC1.

Also disclosed herein are methods of assessing susceptibility of a cancer (such as, for example, a melanoma) in a subject to an immune checkpoint inhibitor therapy comprising: a) obtaining a biological sample (such as, for example, PBMC) from the subject; b) detecting level of NO in the biological sample using flow cytometry; c) measuring immune cell subtype (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)) and NO level in the sample; wherein the immune cell subtypes are immune suppressive cells, or immune effector cells; d) analyzing the nitration of proteins associated with antigen presentation in the sample using LC-MS/MS; wherein increased NO level in immune suppressive cells and/or increased level of nitration of proteins in immune suppressive cells indicate poor immune checkpoint inhibitor therapy response in the subject; and wherein an increased level of NO level in immune effector cells indicates prolonged progression free survival (PFS); and e) administering NO inhibitor (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate) in combination with the immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma if NO is higher in immune suppressive cells. In some aspects, the NO inhibitor is administered on day 2 and administration of immune checkpoint inhibitor therapy on day 0.

In one aspect, disclosed herein are methods of treating, inhibiting, decreasing, reducing, and/or ameliorating an unresectable stage III/IV melanoma in a subject resistant to immune checkpoint inhibitor therapy, comprising: a) measuring chronic NO levels after one year in a biological sample (such as, for example, PBMC) from the subject using a multi-parameter phenotyping tool in R (MPATR) assay; wherein increased NO levels indicate shorter PFS and poor response to immune checkpoint inhibitor therapy alone; and b) administering combination of NO inhibitor (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate) and immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma to the subject if NO level is increased in immune suppressive cells (such as, for example, myeloid-derived suppressor cells (MDSC), regulatory T cells (Tregs), or tumor macrophages). In some aspects, the NO inhibitor is administered on day 2 after the second dose of immune checkpoint inhibitor therapy.

Also disclosed herein are methods of inhibiting epithelial to mesenchymal transition (EMT) in melanoma patients by administering combination of immune 1) checkpoint inhibitors (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma and 2) NO inhibitors (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate).

In one aspect, disclosed herein are composite models to determine a progression free survival (PFS) of a subject with a cancer, wherein the composite model comprises: a) obtaining a biological sample (such as, for example, PBMC) from the subject; b) measuring immune cell subtype; wherein the immune cell subtype are immune suppressive cells, or immune effector cells; c) measuring the nitration of proteins in the sample; wherein increased nitration of proteins in the immune suppressive cells indicate shorter PFS of the subject; and d) determining the subject's response to immune checkpoint inhibitor therapy; wherein an increased nitration of proteins in the immune suppressive cells is an indication of poor response to immune checkpoint inhibitor therapy.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows multiomics (mRNA/proteomics) demonstrates antigen presentation pathways important for anti-PD1 response. The red circles demonstrate the nodes (proteins/mRNA) that are elevated in patients who have >1 year PFS to anti-PD1 therapy when compared with patients with shorter PFS.

FIGS. 2A, 2B, 2C and 2D show MDSCs decrease STAT1 dependent antigen presentation from DCs to CD4+ T cells in a NO dependent manner. FIG. 2A shows the effect of NO on DC function was evaluated using transgenic mice that express a T cell receptor (TCR) specific for a defined antigen, ovalbumin (OVA). DCs and CD4⁺ T cells (ratio 1:4) purified from the spleen and lymph nodes of an OT-II mouse were treated with anti-CD3 antibody (T cell stimulation), the OVA 329-337 peptide (antigen presentation), or whole OVA protein (antigen processing and presentation), LPS (control for inflammation) in the presence or absence of a nitric oxide donor SNAP and analyzed via a [³H]-thymidine incorporation assay of the proliferating OT-II T cells. For space constraints only the OVA peptide stimulus is depicted in B and C. FIG. 2B shows STAT1 deficient DCs markedly impaired the ability of the T cell to recognize the peptide antigen on the surface of the DC and proliferate. FIG. 2C shows MDSCs inhibited antigen presentation and this effect was abrogated in the presence of two iNOS inhibitors (NCX-4016 and L-NAME). FIG. 2D shows purified MDSCs obtained from a tumor bearing mouse was co-cultured with DCs. The DCs were subsequently purified via a magnetic separation process and demonstrated nitrated STAT1 in DCs that were co-cultured with MDSCs.

FIG. 3 shows iNOS inhibitor NCX-4016 increases the efficacy of anti-PD1 blockade in a PD-1 refractory B16 melanoma model. Anti-PD-1 decreases iNOS expression in murine melanoma in a time dependent fashion.

FIG. 4 shows the cytotoxicity of target B16 melanoma cells coated with gp-100 by effector PMEL CD8 T cells is reduced by 100 μM NO donor (SNAP) (*p=0.02). Cytoxicity was measured via flow cytometry with CellTrace™ Far Red (B16 cells) and a live/dead marker (Zombie™ Green).

FIG. 5 shows that levels of nitrated STAT1 increase in the peripheral blood of metastatic melanoma patients as measured by a mass spectrometry assay who are progressing on therapy (red box) with 5 additional data points since 2017. A ratio greater than 0.2 discriminates normal donors from melanoma patients (p=0.039, Fisher Exact Test). Patients with a ratio >0.4 had progressive disease at the time of the blood collection with 5 additional data points (4 melanoma >0.4) and one control <0.1).

FIG. 6 shows that partial least square score predicts long/short relapse-free survival. Kaplan-Meier plot is based on partial least square (PLS) score of lymphocyte phenotypes important for response to anti-CTLA-4 therapy. The PLS score is comprised of 8 phenotypes selected for their nitric oxide content. It is the goal to use the ECOG 1609 samples to measure the nitromics and phosphoproteomics of these samples and associate them with the immune phenotype as measured by flow cytometry.

FIG. 7 shows phenotypic tree demonstrating the distribution of cell phenotypes important for prolonged or short relapse-free survival after anti-CTLA-4 therapy. Each of these phenotypes are characterized for their levels of nitric oxide production. The new data collected in this study can be superimposed on this tree to determine whether the partial least square score may be used to distinguish patients who can experience long vs short relapse-free survival.

FIGS. 8A, 8B, 8C, and 8D shows development and integration of the Multi-Dimensional Phenotyping Analysis Tool in R (MPATR) clustering algorithm to phenotype clinical samples. FIG. 8A shows clustering of immune cell phenotypes. In this iteration, we used the SPADE clustering algorithm. Nine lymphoid markers were used to characterize cellular subsets of patient samples based of median fluorescence intensity. FIG. 8B shows visualization of clustering. Violin plots were constructed, with positive/negative cutoffs for each of the markers. The application can display the violin plots for each node (cluster) or for each sample. In addition, the application can scale the violin plots to the number of events in the node/sample. Each row is labelled by the node/sample number and the number of events in that node/sample. The output is placed in an excel table, with the number of events in each node (phenotype) associated with a relapse-free survival. Statistical analyses may be performed on this reduced dataset, in which each node that can consist of several markers is reduced in dimension to the number of the node. The visualization tool can assist to find populations in conventional flow cytometry software for further visualization. Based upon these analyses, further samples may be collected for hypothesis testing. FIG. 8C shows phenotype dimension reduction. Illustrated is the schema for the number of events per node vs patient sample. FIG. 8D shows schema of the MPATR algorithm. After the phenotype dimension reduction where a multi-parameter flow cytometry stain is reduced to the number of events in a node for a particular sample, statistics may be utilized to determine which nodes are associated with the best or worse response to therapy. Populations are visualized in conventional flow cytometry software and new samples are collected for hypothesis testing.

FIGS. 9A and 9B show example of immune cell type where NO decreases in responders but increases in non-responders to anti-PD-1 therapy (MPATR).

FIG. 10 shows that MDSCs containing NO decrease in responders but remain constant in a non-responder. HLA-DRneg cells gated for CD33 and CD11b (MDSCs).

FIG. 11 shows a phenotype map from multiparameter immunofluorescence demonstrate that immune cells infiltrate tumors that are responsive to anti-PD-1, but in non-responsive tumors, not only is there minimal immune cell infiltration but there is contiguous iNOS expression and minimal expression of PD-L1 (natural ligand for PD-1 that is upregulated in the presence of interferon stimulation) on SOX10+ melanoma cells in close proximity to immune cells. The issues with this sort of analysis is that it requires the setting of a ±threshold for the phenotype map. Work imported data files into MPATR and performed a nearest neighbor analysis on phenotypes (nodes). If the # of nodes=200, then 5 phenotypes >4 different types of phenotypes in close proximity.

FIG. 12 shows PD-L1+/SOX10+ melanoma cells in proximity to T cells (10 FFPE sections from metastatic melanoma patients prior to anti-PD-1 therapy) in the tumor microenvironment is markedly elevated in patients responding to therapy. (p<0.001; unpaired T test). In the right panel, 3 data points are not depicted on this graph (1 or 0 prior to log 10 transformation).

FIG. 13 shows that normal donor human PBMC treated with a nitric oxide donor SNAP have elevated levels of nitrated STAT1 as measured by immunoblot after nitrated tyrosine immunoprecipitation.

FIG. 14 shows elevation in pSTAT1 levels (measured via flow cytometry) in peripheral blood mononuclear cell samples was associated with decreased RFS.

FIG. 15 shows Increases in [nitrated STAT1 (post)] minus [nitrated-STAT1 (pre)] as measured by mass spectrometry predicts for prolonged relapse-free survival to anti-CTLA-4 therapy among melanoma patients undergoing adjuvant ipilimumab treatment.

FIGS. 16A, 16B, and 16C show peripheral blood mononuclear cells (PBMCs) from patients with long relapse-free survival (RFS) displays a small variance of levels of phosphorylated STAT1 (pSTAT1). PBMCs samples from patients were stratified into RFS terciles before anti-CTLA-4 therapy. Tercile 1 represents the poor outcome, whereas tercile 3 represents the favorable outcome following anti-CTLA-4 therapy. (A) pSTAT1 level measured before ipilimumab administration; (B) pSTAT1 level measured after 150 days of adjuvant therapy. Error bar showing ±standard error of the mean (SEM). Abbreviation: MFI, Mean Fluorescence Intensity. (C) Table of number of samples with MFI greater than or less than 1065.

FIGS. 17A and 17B show the Kaplan-Meier relapse-free survival estimates by phosphorylated STAT1 (pSTAT1) level (A) before and (B) after ipilimumab treatment.

FIGS. 18A, 18B, and 18C show the intermediate interferon-α stimulation resulted in the correlative relationship of phosphorylated STAT1 (pSTAT1) levels pre vs post ipilimumab treatment (A) Without interferon-α; (B) with interferon-α stimulation, 10² U/mL; and (C) with interferon-α stimulation, 10⁴ U/mL.

FIG. 19 shows the Change in the magnitude of nitration of STAT1 influences relapse-free survival (RFS) following ipilimumab treatment. Kaplan-Meier survival estimates by change in nSTAT1 concentration, stratified by the median (>−0.004, low n=15, high n=18.

FIGS. 20A, 20B, and 20C show Interferon-alpha stimulation of normal donors PBMCs. FIG. 20A shows the histogram illustrating pSTAT1 expression with different stimulations of Interferon-alpha. FIG. 20B shows the bar chart of mean fluorescence intensity with different amounts of Interferon-alpha to show the dose-dependent activation of pSTAT1 demonstrating saturation at 103 U/mL. FIG. 20C shows the bar chart of mean fluorescence intensity of pSTAT1 with different amounts of Interferon-alpha illustrating different levels of pSTAT1 stimulation in a second normal donor.

FIGS. 21A, 21B, 21C, and 21D show the Kaplan-Meier survival estimate by pSTAT1 level before (FIGS. 21A and 21B) and after (FIGS. 21C and 21D) ipilimumab treatment. PBMCs were treated with 10² U/mL Interferon-alpha stimulation (FIGS. 21A and 21B) and 10⁴ U/mL Interferon-alpha stimulation (FIGS. 21C and 21D).

FIGS. 22A, 22B, and 22C show the paired pSTAT1 level pre versus post ipilimumab treatment without (FIG. 22A) Interferon-alpha stimulation, after (FIG. 22B) 10² U/mL Interferon-alpha stimulation, and (FIG. 22C) 10⁴ U/mL Interferon-alpha stimulation. There is a statistically significant increase (t-test, p<0.01) in the levels of STAT1 in the 10 mg/kg cohort.

FIGS. 23A and 23B show the total nSTAT1 levels before and after ipilimumab did not affect RFS. Kaplan-Meier survival estimate by nSTAT1 concentration before (FIG. 23A) and after (FIG. 23B) ipilimumab treatment.

FIG. 24 shows measurement of generation of NO by suppressor cells and localization of NO (DAFFM) to effector cells within the tumor microenvironment putatively causing inhibition of antigen presentation priming and downstream T cell function. Panel 1 includes (Lymphoid: iNOS, eNOS nNOS, CD45, CD140a, CD3, CD4, CD8, CD11c, CD335, CD25, CD127, CD19, Live-dead marker Myeloid: iNOS, eNOS, nNOS, CD140a, CD45, Ly6G, Ly6C, CD11c, CD11b, CD103 Live Dead marker), Panel 2 includes (lymphoid: DAFFM, CD3, CD8, CD4, CD11c, CD335, CD4, CD25, CD19, CD127myeloid: DAF-FM, CD45, CD11c, CD11b, CD103, Ly6C1, Ly6G1, live dead).

FIGS. 25A and 25B show decreased proliferation with NO donor. FIG. 25A, MTT assay of B16 cells (Left) and YUMMER V600E (Right) demonstrating decreased proliferation with NO donor. FIG. 25B demonstrates iNOS WT mice expression of iNOS in B16 tumors (IHC), whereas iNOS−/− mice do not express iNOS.

FIG. 26 shows frozen human leukapheresis samples demonstrate decrease proliferation in presence of stimulus peptide and can measure recognition of Tetanus Toxoid Dextramer. Left: NO donor (SNAP) decreases proliferation of T cells after antigen presentation of tetanus toxoid (1 ug/ul) from human DC to T cells in a PBMC sample. Right: Measurement of tetramer toxoid dextramer from HLADRB1*0101 cells.

FIG. 27 shows targets for nitration inside of melanoma tumors. Murine B16 tumors were subjected to Phosphotyrosine Proteomics and Expression Proteomics. Compared the differences between anti-PD1 and IgG on multiple days (PBMC collected on day 0 prior to anti-PD1). When compared phosphoproteomics from day 3 to 6, there is increased growth of tumors, mesenchymal processes were elevated.

FIG. 28 shows nitration of NFkB, HDAC1 and ERK1, Day 6 after anti-PD1 exposure from whole tumor lysates.

FIG. 29 depicts nitration of NF-κB increases by Day 3 after anti-PD1 blockade.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term biological sample refers to any portion of biological material from a subject to be used in any of the methods or as a part of any of the compositions disclosed herein including, but are limited to tissue biopsy, whole blood, peripheral blood mononuclear cells (PBMC), urine sample, lung lavage, sputum, saliva, and fecal sample. The biological can include samples for normal and cancerous tissue. Sample may be obtained from any tissue a subject by any means known in the art (tissue resection, biopsy phlebotomy, core biopsy).

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. In particular, the immune checkpoint inhibitor of the present invention is administered for enhancing the proliferation, migration, persistence and/or cytoxic activity of CD8+ T cells in the subject and in particular the tumor-infiltrating of CD8+ T cells of the subject.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years. In some embodiments, the regimen is determined to achieve prolonged progression free survival of at least 6 months. In some embodiments, the prolonged progression free survival is at least 7 months. In some embodiments, the prolonged progression free survival is at least 8 months. In some embodiments, the prolonged progression free survival is at least 9 months. In some embodiments, the prolonged progression free survival is at least 10 months. In some embodiments, the prolonged progression free survival is at least 11 months. In some embodiments, the prolonged progression free survival is at least 12 months.

As used herein, the term “progression free survival” means the time period for which a subject having a disease (e.g. cancer) survives, without a significant worsening of the disease state. Progression free survival may be assessed as a period of time in which there is no progression of tumor growth and/or wherein the disease status of a patient is not determined to be a progressive disease. In some embodiments, progression free survival of a subject having cancer is assessed by evaluating tumor (lesion) size, tumor (lesion) number, and/or metastasis.

As used herein, “progression free survival” (PFS) is defined as time period from treatment randomization to the earlier date of assessment progression on the next anticancer therapy following study treatment or death by any cause. In some embodiments, determination of progression may be assessed by clinical and/or radiographic assessment.

The term “progression” of tumor growth or a “progressive disease” (PD) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Treating Cancer

The immune system has a critical role in the development, progression, and effective treatment of melanoma. The response rate of single agent anti-PD-1 is approximately 40%, and in combination with anti-CTLA-4 therapy response rates increase to 50-60%, at the cost of significantly increased toxicity. This proposal explores the mechanism(s) of resistance in the 60% of patients who do not respond to single agent anti-PD-1 therapy with the intent to develop new therapeutic approaches and an assay to predict treatment response.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules) Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because the tumor microenvironment has relatively high levels of adenosine, which lead to a negative immune feedback loop through the activation of A2AR. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152 is overexpressed on Treg cells serves to control T cell proliferation. IDO, Indoleamine 2,3-dioxygenase, is a tryptophan catabolic enzyme, a related immune-inhibitory enzymes. Another important molecule is TDO, tryptophan 2,3-dioxygenase. IDO is known to suppress T and NK cells, generate and activate Tregs and myeloid-derived suppressor cells, and promote tumor angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA. Short for V-domain Ig suppressor of T cell activation, VISTA is primarily expressed on hematopoietic cells so that consistent expression of VISTA on leukocytes within tumors may allow VISTA blockade to be effective across a broad range of solid tumors.

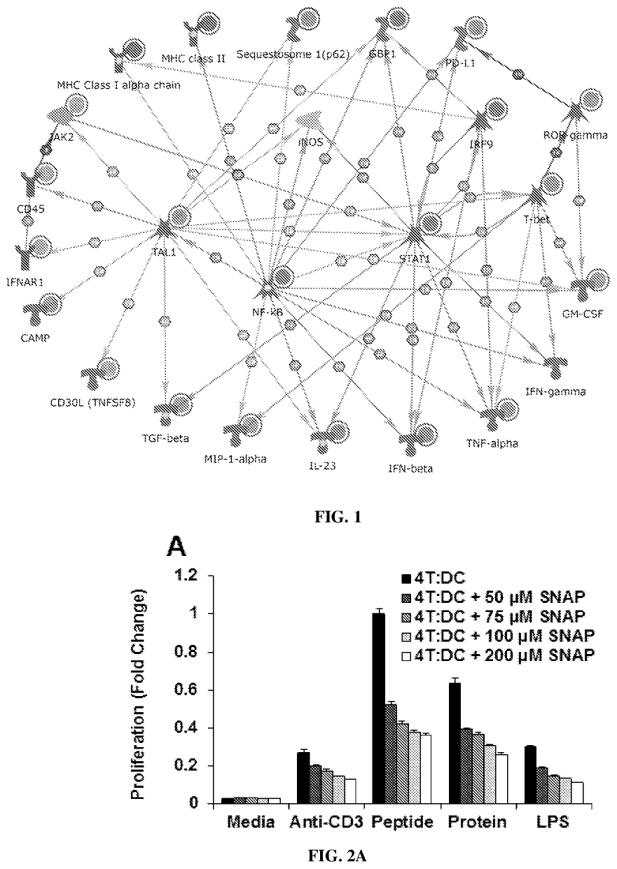

Melanoma cells are recognized and eliminated by the immune system, but this process is abrogated by tumor-mediated mechanisms including production of reactive oxygen and nitrogen species, secretion of immune-suppressive cytokines, and induction of inhibitory immune cells. Classically, antigen presentation to T cells by DCs is defective in melanoma. Stimulation of DCs with type I+II interferons (IFN-α and γ) and downstream signal transduction via the janus kinase-signal transducer and activator of transcription (Jak-STAT) pathway generate effective host T cell immune responses to cancer. Dysregulation of the JAK-STAT pathway have been implicated in anti-PD-1 resistance (rare mutations in the JAK2 protein) and that this effect is likely due to defective IFN signaling to express PD-L1 on the cell surface (FIG. 1). There are clearly other causes of tumor promoting JAK/STAT signaling. Infiltration of immune cells in melanoma tissues are associated with response but are not of sufficient sensitivity/specificity for clinical utility. Furthermore, in DCs, IFN-α+IFN-γ signaling are responsible for upregulation of MHC I and II molecules for the presentation of antigens. In particular, increased MHC class II expression levels in melanomas are associated with response to anti-PD-1 therapy. Antitumor effects of IFN-α are dependent on STAT1 signal transduction in immune cells via phosphorylation of tyrosine 701. JAK-STAT signaling was markedly inhibited in human peripheral blood immune cells from tumor bearing patients. The mechanism of immune inhibition was proposed involves nitric oxide (NO) secretion by tumor-induced inhibitory immune cells (known as myeloid-derived suppressor cells, MDSCs) and decreased p-STAT1 signaling in response to interferon signaling. Production of NO by MDSCs leads to the production of reactive nitrogen species which are chemical entities inside cells derived from NO (e.g. peroxynitrite—ONOO⁻) that cause nitration at key amino acids such as tyrosine. In humans, MDSCs are described by both their functional capacity to suppress T cell activation and immature myeloid phenotype (typically CD33+, CD11b⁺, HLADR^{low/-}). MDSC numbers increase with more advanced stages of melanoma.

There is an increased MDSC population associated with poor anti-PD-1 response in melanoma. Anti-PD-1 effects are dependent on MHC class II activities. NO inhibits antigen presentation from DCs to CD4 T cells in the presence of MDSCs in melanoma and nitrated STAT1 can be found in melanoma specimens. LCK-1, Guanylate Cyclase (Jak3/STAT5 pathway) as well as MHC and TCR molecules are also known to be nitrated and inhibit immune surveillance. FIG. 1 demonstrates antigen presentation pathways important for response to anti-PD1 therapy and interestingly all of these proteins except for those in JAK3/STAT5 pathway and LCK1 appear to be upregulated in mediating responses to anti-PD-1. NFκB is also known to be nitrated and is an important pro-tumor factor. There is a direct interaction between STAT1, NF-κB, and iNOS (FIG. 1) and I plan to study nitration of STAT1, NFkB as well as the other proteins involved in antigen presentation to investigate how inhibition of iNOS can abrogate resistance to checkpoint blockade. Given the discovery that MDSC-derived NO leads to the with nitration of proteins that are crucial for antigen presentation and the generation of an effective host immune response, MDSC-mediated nitration of proteins involved in antigen presentation in DCs and T cells is an important actionable mechanism of immune inhibition in the setting of melanoma and that reversal of this inhibition can lead to improved antitumor immunity in the setting of anti-PD-1 therapy.

Secretion of nitric oxide (NO) by tumor-induced inhibitory immune cells (known as myeloid-derived suppressor cells [MDSCs]) and decreases phospho-STAT1 (pSTAT1) signaling in response to interferon signaling. In melanoma, MDSC numbers increase in patients with poor response to ipilimumab therapy, and levels of NO increase in more advanced stages of melanoma. Although levels of NO in MDSC are associated with response, the difference between the poor and good responder survival curves is not robust enough to provide for a clinical test. Rather, a lymphoid cell signature including the levels of NO may predict response to ipilimumab (anti-CTLA-4) among melanoma patients undergoing adjuvant therapy after resection for stage III/IV melanoma. Furthermore, NO inhibits antigen presentation from DCs to CD4+ T cells. LCK-1 and guanylate cyclase (Jak3/STAT5 pathway), as well as MHC and TCR molecules, are also known to be nitrated and inhibit immune surveillance. NFκB is also known to be nitrated and is an important pro-tumor factor. Shown herein nitration of proteins is an important mechanism of immune inhibition in melanoma to anti-CTLA-4 therapy and that reversal of this inhibition leads to improved antitumor immunity.

In one aspect, disclosed herein are methods of reducing immune checkpoint inhibitor resistance comprising administering a nitric oxide inhibitor (such as, for example, NCX-4016, L-nitroarginine methyl ester (L-NAME), Aminoguanidine hydrochloride, S-Isopropylisothiourea hydrobromide, or (S)-Methylisothiourea sulfate) to a subject being treated for a disease (such as, for example, a cancer, including, but not limited to melanoma) with an immune checkpoint inhibitor (such as, for example, antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

In one aspect, disclosed herein are methods of reducing immune checkpoint inhibitor resistance wherein the nitric oxide inhibitor is administered no greater than 3 days after administration of the immune checkpoint inhibitor. In one aspect the nitric oxide inhibitor is administered at least 1, 2, 3, 4, 5 6, 7, 8, 9 10, 11, 12, 13, 14, 15, 16, 17, 18, 19, 20, 21, 22, 23, 24, 25, 26, 27, 28, 29, 30, 31, 60, 90 days prior to administration of or 5, 10, 15, 20, 25, 30, 35, 40, 45, 50, 55, 60, 75, 90, 105, 120, 150, 180 min, 4, 5, 6, 7, 8, 9, 10, 11, 12, 13, 14, 15, 16, 17, 18, 19, 20, 21, 22, 23, 24, 36, 48, 60, or 72 after administration of the immune checkpoint inhibitor.

The disclosed methods can reduce resistance of a disease to any immune checkpoint inhibitor known in the art including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

As the disclosed methods can reduce resistance of a disease (such as a cancer) to an immune checkpoint inhibitor, it is understood and herein contemplated that the nitric oxide inhibitor and the immune checkpoint inhibitor can be used in combination to treat a disease (such as a cancer). Accordingly, disclosed herein are combinations therapy comprising a nitric oxide inhibitor (such as, for example, NCX-4016, L-nitroarginine methyl ester (L-NAME), Aminoguanidine hydrochloride, S-Isopropylisothiourea hydrobromide, or (S)-Methylisothiourea sulfate) and an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

Also disclosed herein are methods of treating, reducing, inhibiting, decreasing, ameliorating, and/or preventing a disease (such as, for example, a caner and/or metastasis including, but not limited to melanoma) in a subject comprising administering to the subject a nitric oxide inhibitor (such as, for example, NCX-4016, L-nitroarginine methyl ester (L-NAME), Aminoguanidine hydrochloride, S-Isopropylisothiourea hydrobromide, or (S)-Methylisothiourea sulfate) and an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

In one aspect, disclosed herein are methods of detecting resistance of a disease or condition (such as for example, cancer or immune checkpoint inhibitor toxicity) in a subject to an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep) comprising a) obtaining a tissue sample from a subject that is receiving or that is likely to receive an immune checkpoint inhibitor for treatment of a disease or condition; b) measuring the level of nitration in the tissue sample (such as, for example, nitration measured by one or more of mass spectroscopy, flow cytometry, immunohistochemical staining, immunoblot, Multi-Dimensional Phenotyping Analysis Tool in R (MPATR) clustering, and/or proteomics); wherein an increase in nitration relative to a control indicates that the disease or condition is resistant to an immune checkpoint inhibitor; and c) when a subject has a disease or condition that is resistant to an immune checkpoint inhibitor, administering to the subject a nitric oxide inhibitor (such as, for example, nitic oxide donating compounds (for example nitroaspirin (e.g NCX-4016), NOSH-Aspirin), L-nitroarginine methyl ester (L-NAME), NG-monomethyl-1-arginine (L-NMMA), Aminoguanidine hydrochloride, S-Isopropylisothiourea hydrobromide, or (S)-Methylisothiourea sulfate).

In one aspect, disclosed herein are methods of treating, inhibiting, decreasing, reducing, and/or ameliorating an unresectable stage III/IV melanoma in a subject resistant to immune checkpoint inhibitor therapy, comprising: a) measuring chronic NO levels after one year in a biological sample (such as, for example, PBMC) from the subject using a multi-parameter phenotyping tool in R (MPATR) assay; wherein increased NO levels indicate shorter PFS and poor response to immune checkpoint inhibitor therapy alone; and b) administering combination of NO inhibitor (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate) and immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma to the subject if NO level is increased in immune suppressive cells (such as, for example, myeloid-derived suppressor cells (MDSC), regulatory T cells (Tregs), or tumor macrophages). In some aspects, the NO inhibitor is administered on day 2 after the second dose of immune checkpoint inhibitor therapy.

It is understood and herein contemplated that the combinations of an immune checkpoint inhibitor and nitric oxide inhibitor can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, pancreatic cancer, sarcoma, or melanoma.

The disclosed treatment methods are not limited to the use of the disclosed immune checkpoint inhibitors and can include any anti-cancer therapy known in the art including, but not limited to Abemaciclib, Abiraterone Acetate, ABITREXATE® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, ADRIAMYCIN® (Doxorubicin Hydrochloride), Afatinib Dimaleate, AFINITOR® (Everolimus), AKYNZEO® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), ALOXI® (Palonosctron Hydrochloride), ALUNBRIG® (Brigatinib), AMBOCHLORIN® (Chlorambucil), AMBOCLORIN® (Chlorambucil), Amifostine, Aminolevulinic Acid, Anastrozole, Aprepitant, AREDIA® (Pamidronate Disodium), ARIMIDEX® (Anastrozole), AROMASIN® (Exemestane), ARRANON® (Nelarabine), Arsenic Trioxide, ARZERRA® (Ofatumumab), Asparaginase Erwinia chrysanthemi, Atezolizumab, AVASTIN® (Bevacizumab), Avelumab, Axitinib, Azacitidine, BAVENCIO® (Avelumab), BEACOPP, BECENUM® (Carmustine), BELEODAQ® (Belinostat), Belinostat, Bendamustine Hydrochloride, BEP, BESPONSA® (Inotuzumab Ozogamicin), Bevacizumab, Bexarotene, BEXXAR® (Tositumomab and Iodine I 131 Tositumomab), Bicalutamide, BICNU® (Carmustine), Bleomycin, Blinatumomab, BLINCYTO® (Blinatumomab), Bortezomib, BOSULIF® (Bosutinib), Bosutinib, Brentuximab Vedotin, Brigatinib, BuMel, Busulfan, BUSULFEX® (Busulfan), Cabazitaxel, CABOMETYX® (Cabozantinib-S-Malate), Cabozantinib-S-Malate, CAF, CAMPATH® (Alemtuzumab), CAMPTOSAR® (Irinotecan Hydrochloride), Capecitabine, CAPOX, CARAC® (Fluorouracil—Topical), Carboplatin, CARBOPLATIN-TAXOL, Carfilzomib, CARMUBRIS® (Carmustine), Carmustine, Carmustine Implant, CASODEX® (Bicalutamide), CEM, Ceritinib, CERUBIDINE® (Daunorubicin Hydrochloride), CERVARIX® (Recombinant HPV Bivalent Vaccine), Cetuximab, CEV, Chlorambucil, CHLORAMBUCIL-PREDNISONE, CHOP, Cisplatin, Cladribine, CLAFEN® (Cyclophosphamide), Clofarabine, CLOFAREX® (Clofarabine), CLOLAR® (Clofarabine), CMF, Cobimetinib, COMETRIQ® (Cabozantinib-S-Malate), Copanlisib Hydrochloride, COPDAC, COPP, COPP-ABV, COSMEGEN® (Dactinomycin), COTELLIC® (Cobimetinib), Crizotinib, CVP, Cyclophosphamide, CYFOS® (Ifosfamide), CYRAMZA® (Ramucirumab), Cytarabine, Cytarabine Liposome, CYTOSAR-U® (Cytarabine), CYTOXAN® (Cyclophosphamide), Dabrafenib, Dacarbazine, DACOGEN® (Decitabine), Dactinomycin, Daratumumab, DARZALEX® (Daratumumab), Dasatinib, Daunorubicin Hydrochloride, Daunorubicin Hydrochloride and Cytarabine Liposome, Decitabine, Defibrotide Sodium, DEFITELIO® (Defibrotide Sodium), Degarelix, Denileukin Diftitox, Denosumab, DEPOCYT® (Cytarabine Liposome), Dexamethasone, Dexrazoxane Hydrochloride, Dinutuximab, Docetaxel, DOXIL® (Doxorubicin Hydrochloride Liposome), Doxorubicin Hydrochloride, Doxorubicin Hydrochloride Liposome, DOX-SL® (Doxorubicin Hydrochloride Liposome), DTIC-DOME® (Dacarbazine), Durvalumab, EFUDEX® (Fluorouracil—Topical), ELITEK® (Rasburicasc), ELLENCE® (Epirubicin Hydrochloride), Elotuzumab, ELOXATIN® (Oxaliplatin), Eltrombopag Olamine, EMEND® (Aprepitant), EMPLICITI® (Elotuzumab), Enasidenib Mesylate, Enzalutamide, Epirubicin Hydrochloride, EPOCH, ERBITUX® (Cetuximab), Eribulin Mesylate, ERIVEDGE® (Vismodegib), Erlotinib Hydrochloride, ERWINAZE® (Asparaginase Erwinia chrysanthemi), ETHYOL® (Amifostine), Etopophos ETOPOPHOS® (Etoposide Phosphate), Etoposide, Etoposide Phosphate, EVACET® (Doxorubicin Hydrochloride Liposome), Everolimus, EVISTA® (Raloxifene Hydrochloride), EVOMELA® (Melphalan Hydrochloride), Exemestane, 5-FU® (Fluorouracil Injection), 5-FU® (Fluorouracil—Topical), FARESTON® (Toremifene), FARYDAK® (Panobinostat), FASLODEX® (Fulvestrant), FEC, FEMARA® (Letrozole), Filgrastim, FLUDARA® (Fludarabine Phosphate), Fludarabine Phosphate, FLUOROPLEX® (Fluorouracil—Topical), Fluorouracil Injection, Fluorouracil—Topical, Flutamide, FOLEX® (Methotrexate), FOLEX PFS® (Methotrexate), FOLFIRI, FOLFIRI-BEVACIZUMAB, FOLFIRI-CETUXIMAB, FOLFIRINOX, FOLFOX, FOLOTYN® (Pralatrexate), FU-LV, Fulvestrant, GARDASIL® (Recombinant HPV Quadrivalent Vaccine), GARDASIL 9® (Recombinant HPV Nonavalent Vaccine), GAZYVA® (Obinutuzumab), Gefitinib, Gemcitabine Hydrochloride, GEMCITABINE-CISPLATIN, GEMCITABINE-OXALIPLATIN, Gemtuzumab Ozogamicin, GEMZAR® (Gemcitabine Hydrochloride), GILOTRIF® (Afatinib Dimaleate), GLEEVEC® (Imatinib Mesylate), GLIADEL® (Carmustine Implant), GLIADEL WAFER® (Carmustine Implant), Glucarpidase, Goserelin Acetate, HALAVEN® (Eribulin Mesylate), HEMANGEOL® (Propranolol Hydrochloride), HERCEPTIN® (Trastuzumab), HPV Bivalent Vaccine, Recombinant, HPV Nonavalent Vaccine, Recombinant, HPV Quadrivalent Vaccine, Recombinant, HYCAMTIN® (Topotecan Hydrochloride), HYDREA® (Hydroxyurea), Hydroxyurea, Hyper-CVAD, IBRANCE® (Palbociclib), Ibritumomab Tiuxetan, Ibrutinib, ICE, ICLUSIG® (Ponatinib Hydrochloride), IDAMYCIN® (Idarubicin Hydrochloride), Idarubicin Hydrochloride, Idelalisib, IDHIFA® (Enasidenib Mesylate), IFEX® (Ifosfamide), Ifosfamide, IFOSFAMIDUM® (Ifosfamide), IL-2 (Aldesleukin), Imatinib Mesylate, IMBRUVICA® (Ibrutinib), IMFINZI® (Durvalumab), Imiquimod, IMLYGIC® (Talimogene Laherparepvec), INLYTA® (Axitinib), Inotuzumab Ozogamicin, Interferon Alfa-2b, Recombinant, Interleukin-2 (Aldesleukin), INTRON A® (Recombinant Interferon Alfa-2b), Iodine I 131 Tositumomab and Tositumomab, Ipilimumab, IRESSA® (Gefitinib), Irinotecan Hydrochloride, Irinotecan Hydrochloride Liposome, ISTODAX® (Romidepsin), Ixabepilone, Ixazomib Citrate, IXEMPRA® (Ixabepilone), JAKAFI® (Ruxolitinib Phosphate), JEB, JEVTANA® (Cabazitaxel), KADCYLA® (Ado-Trastuzumab Emtansine), KEOXIFENE® (Raloxifene Hydrochloride), KEPIVANCE® (Palifermin), KEYTRUDA® (Pembrolizumab), KISQALI® (Ribociclib), KYMRIAH® (Tisagenlecleucel), KYPROLIS® (Carfilzomib), Lanreotide Acetate, Lapatinib Ditosylate, LARTRUVO® (Olaratumab), Lenalidomide, Lenvatinib Mesylate, LENVIMA® (Lenvatinib Mesylate), Letrozole, Leucovorin Calcium, LEUKERAN® (Chlorambucil), Leuprolide Acetate, LEUSTATIN® (Cladribine), LEVULAN® (Aminolevulinic Acid), LINFOLIZIN® (Chlorambucil), LIPODOX® (Doxorubicin Hydrochloride Liposome), Lomustine, LONSURF® (Trifluridine and Tipiracil Hydrochloride), LUPRON® (Leuprolide Acetate), LUPRON DEPOT® (Leuprolide Acetate), LUPRON DEPOT-PED® (Leuprolide Acetate), LYNPARZA® (Olaparib), MARQIBO® (Vincristine Sulfate Liposome), MATULANE® (Procarbazine Hydrochloride), Mechlorethamine Hydrochloride, Megestrol Acetate, MEKINIST® (Trametinib), Melphalan, Melphalan Hydrochloride, Mercaptopurine, Mesna, MESNEX® (Mesna), METHAZOLASTONE® (Temozolomide), Methotrexate, METHOTREXATE LPF® (Methotrexate), Methylnaltrexone Bromide, MEXATE® (Methotrexate), MEXATE-AQ® (Methotrexate), Midostaurin, Mitomycin C, Mitoxantrone Hydrochloride, MITOZYTREX® (Mitomycin C), MOPP, MOZOBIL® (Plerixafor), MUSTARGEN® (Mechlorethamine Hydrochloride), MUTAMYCIN® (Mitomycin C), MYLERAN® (Busulfan), MYLOSAR® (Azacitidine), MYLOTARG® (Gemtuzumab Ozogamicin), NANOPARTICLE PACLITAXEL® (Paclitaxel Albumin-stabilized Nanoparticle Formulation), NAVELBINE® (Vinorelbine Tartrate), Necitumumab, Nelarabine, NEOSAR® (Cyclophosphamide), Neratinib Maleate, NERLYNX® (Neratinib Maleate), Netupitant and Palonosetron Hydrochloride, NEULASTA® (Pegfilgrastim), NEUPOGEN® (Filgrastim), NEXAVAR® (Sorafenib Tosylate), NILANDRON® (Nilutamide), Nilotinib, Nilutamide, NINLARO® (Ixazomib Citrate), Niraparib Tosylate Monohydrate, Nivolumab, NOLVADEX® (Tamoxifen Citrate), NPLATE® (Romiplostim), Obinutuzumab, ODOMZO® (Sonidegib), OEPA, Ofatumumab, OFF, Olaparib, Olaratumab, Omacetaxine Mepesuccinate, ONCASPAR® (Pegaspargase), Ondansetron Hydrochloride, ONIVYDE® (Irinotecan Hydrochloride Liposome), ONTAK® (Denilcukin Diftitox), OPDIVO® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, PARAPLAT® (Carboplatin), PARAPLATIN® (Carboplatin), Pazopanib Hydrochloride, PCV, PEB, Pegaspargase, Pegfilgrastim, Peginterferon Alfa-2b, PEG-INTRON® (Peginterferon Alfa-2b), Pembrolizumab, Pemetrexed Disodium, PERJETA® (Pertuzumab), Pertuzumab, PLATINOL® (Cisplatin), PLATINOL-AQ® (Cisplatin), Plerixafor, Pomalidomide, POMALYST® (Pomalidomide), Ponatinib Hydrochloride, PORTRAZZA® (Necitumumab), Pralatrexate, Prednisone, Procarbazine Hydrochloride, PROLEUKIN® (Aldesleukin), PROLIA® (Denosumab), PROMACTA® (Eltrombopag Olamine), Propranolol Hydrochloride, PROVENGE® (Sipuleucel-T), PURINETHOL® (Mercaptopurine), PURIXAN® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, RELISTOR® (Methylnaltrexone Bromide), R-EPOCH, REVLIMID® (Lenalidomide), RHEUMATREX® (Methotrexate), Ribociclib, R-ICE, RITUXAN® (Rituximab), RITUXAN HYCELA® (Rituximab and Hyaluronidase Human), Rituximab, Rituximab and, Hyaluronidase Human, Rolapitant Hydrochloride, Romidepsin, Romiplostim, RUBIDOMYCIN® (Daunorubicin Hydrochloride), RUBRACA® (Rucaparib Camsylate), Rucaparib Camsylate, Ruxolitinib Phosphate, RYDAPT® (Midostaurin), Sclerosol Intrapleural Aerosol (Talc), Siltuximab, Sipuleucel-T, SOMATULINE DEPOT® (Lanreotide Acetate), Sonidegib, Sorafenib Tosylate, SPRYCEL® (Dasatinib), STANFORD V, Sterile Talc Powder (Talc), STERITALC® (Talc), STIVARGA® (Regorafenib), Sunitinib Malate, SUTENT® (Sunitinib Malate), SYLATRON® (Peginterferon Alfa-2b), SYLVANT® (Siltuximab), Synribo SYNRIBO® (Omacetaxine Mepesuccinate), TABLOID® (Thioguanine), TAC, TAFINLAR® (Dabrafenib), TAGRISSO® (Osimertinib), Talc, Talimogene Laherparepvec, Tamoxifen Citrate, TARABINE PFS® (Cytarabine), TARCEVA® (Erlotinib Hydrochloride), TARGRETIN® (Bexarotene), TASIGNA® (Nilotinib), TAXOL® (Paclitaxel), TAXOTERE® (Docetaxel), TECENTRIQ® (Atczolizumab), TEMODAR® (Temozolomide), Temozolomide, Temsirolimus, Thalidomide, THALOMID® (Thalidomide), Thioguanine, Thiotepa, Tisagenlecleucel, TOLAK® (Fluorouracil—Topical), Topotecan Hydrochloride, Toremifene, TORISEL® (Temsirolimus), Tositumomab and Iodine I 131 Tositumomab, TOTECT® (Dexrazoxane Hydrochloride), TPF, Trabectedin, Trametinib, Trastuzumab, TREANDA® (Bendamustine Hydrochloride), Trifluridine and Tipiracil Hydrochloride, TRISENOX® (Arsenic Trioxide), TYKERB® (Lapatinib Ditosylate), UNITUXIN® (Dinutuximab), Uridine Triacetate, VAC, Vandetanib, VAMP, VARUBI® (Rolapitant Hydrochloride), VECTIBIX® (Panitumumab), VeIP, VELBAN® (Vinblastine Sulfate), VELCADE® (Bortezomib), VELSAR® (Vinblastine Sulfate), Vemurafenib, VENCLEXTA® (Venetoclax), Venetoclax, VERZENIO® (Abemaciclib), VIADUR® (Leuprolide Acetate), VIDAZA® (Azacitidine), Vinblastine Sulfate, VINCASAR PFS® (Vincristine Sulfate), Vincristine Sulfate, Vincristine Sulfate Liposome, Vinorelbine Tartrate, VIP, Vismodegib, VISTOGARD® (Uridine Triacetate), VORAXAZE® (Glucarpidase), Vorinostat, VOTRIENT® (Pazopanib Hydrochloride), VYXEOS® (Daunorubicin Hydrochloride and Cytarabine Liposome), WELLCOVORIN® (Leucovorin Calcium), XALKORI® (Crizotinib), XELODA® (Capecitabine), XELIRI, XELOX, XGEVA® (Denosumab), XOFIGO® (Radium 223 Dichloride), XTANDI® (Enzalutamide), YERVOY® (Ipilimumab), YONDELIS® (Trabectedin), ZALTRAP® (Ziv-Aflibercept), ZARXIO® (Filgrastim), ZEJULA® (Niraparib Tosylate Monohydrate), ZELBORAF® (Vemurafenib), ZEVALIN® (Ibritumomab Tiuxetan), ZINECARD® (Dexrazoxane Hydrochloride), Ziv-Aflibercept, ZOFRAN® (Ondansetron Hydrochloride), ZOLADEX® (Goserelin Acetate), Zoledronic Acid, ZOLINZA® (Vorinostat), ZOMETA® (Zoledronic Acid), ZYDELIG® (Idelalisib), ZYKADIA® (Ceritinib), and/or ZYTIGA® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

Also disclosed herein are methods of improving, or increasing progression free survival (PFS) of a cancer (including, but not limited to melanoma) subject with a cancer resistant to conventional cancer therapy (including, but not limited to subjects with cancers that are resistant to an immune checkpoint inhibitor therapy (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), Interferon-alpha, Interferon-gamma)), the method comprising: obtaining a biological sample (such as, for example, peripheral blood mononuclear cells (PBMC)) from the subject, performing a multi-parameter phenotyping tool in R (MPATR) assay on the biological sample, analyzing the nitric oxide (NO) levels in the biological sample, measuring the immune cells subtype producing NO (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)), and administering a NO inhibitor (such as, for example, nitic oxide donating compounds (for example nitroaspirin (e.g NCX-4016), NOSH-Aspirin), L-nitroarginine methyl ester (L-NAME), L-NMMA, Aminoguanidine hydrochloride, S-Isopropylisothiourea hydrobromide, or (S)-Methylisothiourea sulfate) in combination with an immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma to subjects with a cancer resistant to conventional cancer therapy having increased levels of NO in immune suppressive cells. In some aspects, the NO inhibitor is administered on day 2 and administration of immune checkpoint inhibitor therapy on day 0.

In one aspect, disclosed herein are multi-parameter phenotyping tool in R (MPATR) assay to identify nitration of proteins in an immune cell subtype, comprising: a) obtaining biological sample (such as, for example, peripheral blood mononuclear cells (PBMC)) from a subject with a cancer (such as, for example, melanoma) that is resistant to conventional cancer therapy; b) measuring the immune cell subtype (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)) producing NO; wherein the immune cell subtype are immune suppressive cells or immune effector cells; and c) measuring the nitration of proteins in the immune cells (including, but not limited to measuring nitration using mass spectrometry); wherein an increased level of nitration of proteins in immune suppressive cells is an indication that the subject needs a NO inhibitor (such as, for example, nitic oxide donating compounds (for example nitroaspirin (e.g NCX-4016), NOSH-Aspirin), L-nitroarginine methyl ester (L-NAME), L-NMMA, Aminoguanidine hydrochloride, S-Isopropylisothiourea hydrobromide, or (S)-Methylisothiourea sulfate) to overcome resistance to conventional cancer therapy (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma.

Also disclosed herein are MPATR assays, wherein the subtype of the immune cells is measured using flow cytometry panels consisting of a myeloid cell panel, a lymphoid cell panel, and a secondary panel; wherein the panels were stained with myeloid antibodies (such as for example, antibodies that bind DAF-FM, HLA-DR-PE-Cy7, CD33-APC, CD11b-BV421, CD14-BUV395, CD15-BV510, or CD11c-PE), or lymphoid antibodies (such as, for example, antibodies that bind DAF-FM, CD3-BUV395, CD8-BV510, CD11c-PE, CD56-BV421, CD4-AF700, CD19-PE-Dazzle, CD25-PE-Cy7, or CD127-APC). In some aspects, the panels can further comprise a secondary panel of antibodies, including, but not limited to antibodies that bind IFNy, PD-L1, CTLA4, Arginase 1, FoxP3, TCR-ζ, or CD69.

In one aspect, disclosed herein are MPATR assays, wherein the proteins identified for nitration comprises STAT1, Actin, NF-kB, STAT3, PUS7, FAK1, or HDAC1.

Also disclosed herein are methods of assessing susceptibility of a cancer (such as, for example, a melanoma) in a subject to an immune checkpoint inhibitor therapy comprising: a) obtaining a biological sample (such as, for example, PBMC) from the subject; b) detecting level of NO in the biological sample using flow cytometry; c) measuring immune cell subtype (such as, for example, immune suppressive cells (including, but not limited to myeloid-derived suppressor cells (MDSC), regulatory T cells (Tregs), and tumor macrophage) or immune effector cells (including, but not limited to cytotoxic CD8+ T cells (including, but not limited to effector CD8 T cells, peripheral memory CD8 T cells, central memory CD8 T cells), helper CD4 T cells, memory CD4 T cells, natural killer (NK) cells, NK T cells, B cells, plasma cells, and dendritic cells)) and NO level in the sample; wherein the immune cell subtypes are immune suppressive cells, or immune effector cells; d) analyzing the nitration of proteins associated with antigen presentation in the sample using LC-MS/MS; wherein increased NO level in immune suppressive cells and/or increased level of nitration of proteins in immune suppressive cells indicate poor immune checkpoint inhibitor therapy response in the subject; and wherein an increased level of NO level in immune effector cells indicates prolonged progression free survival (PFS); and e) administering NO inhibitor (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate) in combination with the immune checkpoint inhibitor (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma if NO is higher in immune suppressive cells. In some aspects, the NO inhibitor is administered on day 2 and administration of immune checkpoint inhibitor therapy on day 0.

Also disclosed herein are methods of inhibiting epithelial to mesenchymal transition (EMT) in melanoma patients by administering combination of immune 1) checkpoint inhibitors (including, but not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep), interferon (IFN)-alpha, and/or IFN-gamma and 2) NO inhibitors (such as, for example, NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate).

In one aspect, disclosed herein are composite models to determine a progression free survival (PFS) of a subject with a cancer, wherein the composite model comprises: a) obtaining a biological sample (such as, for example, PBMC) from the subject; b) measuring immune cell subtype; wherein the immune cell subtype are immune suppressive cells, or immune effector cells; c) measuring the nitration of proteins in the sample; wherein increased nitration of proteins in the immune suppressive cells indicate shorter PFS of the subject; and d) determining the subject's response to immune checkpoint inhibitor therapy; wherein an increased nitration of proteins in the immune suppressive cells is an indication of poor response to immune checkpoint inhibitor therapy.

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nitric oxide inhibitor is disclosed and discussed and a number of modifications that can be made to a number of molecules including the nitric oxide inhibitor are discussed, specifically contemplated is each and every combination and permutation of nitric oxide inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### a) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The compositions, including antibodies, can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal, or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases, and the like.

Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone can range from about 1 μg/kg to up to 100 mg/kg of body weight or more per day, depending on the factors mentioned above.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Unless indicated otherwise, parts are parts by weight, temperature is in ° C. or is at ambient temperature, and pressure is at or near atmospheric.

### Example 1: Assess how Anti-PD-1 Resistance in Melanoma Results from NO Inhibiting Dendritic Cell Antigen Presentation to T Cells

### a) NO Causes a Decrease in Antigen Presentation in the Setting of Anti-PD-1 Therapy

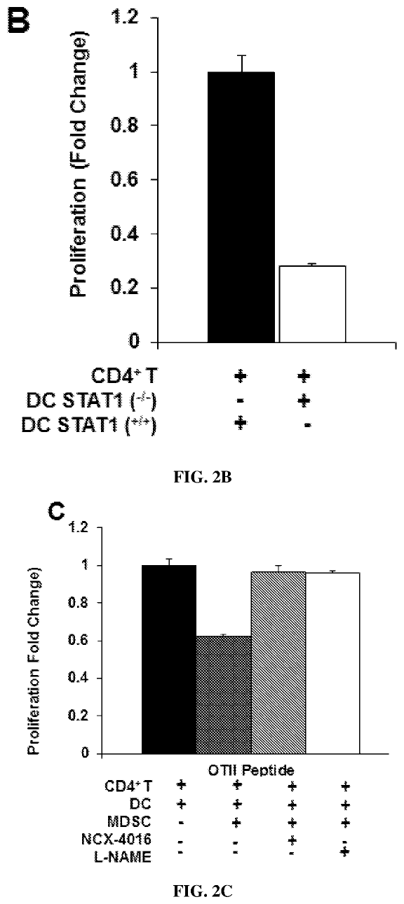

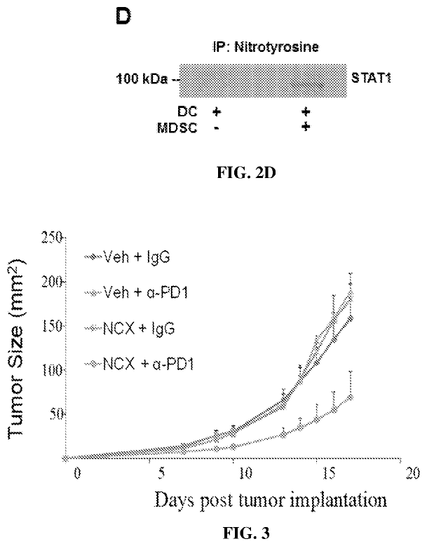

The effects of NO on DC function using transgenic mice were evaluated that express a T cell receptor (TCR) specific for a defined antigen, namely OVA. CD4+ T cells were harvested from the spleens of TCR transgenic OT-II mice whose TCR recognize an OVA peptide (aa 329-337) in the context of MHC II molecules. The data now show that NO can inhibit DC antigen presentation to CD4+ T cells via utilization of the nitric oxide donor SNAP (S-Nitroso-N-Acetyl-D,L-Penicillamine) (FIG. 2A), antigen presentation is markedly impaired in DCs without the STAT1 protein (FIG. 2B), MDSCs derived from B16 melanoma inhibited antigen presentation (FIG. 2C), and MDSCs facilitated nitration of STAT1 in DCs (FIG. 2D). Levels of SNAP or other NO donors such as deta-NONOate can be adjusted to mimic the physiological concentration of NO within MDSCs and be utilized to directly measure binding of tetramers to the TCR complex. To simulate a real world scenario of antigen expressed naturally by the B16 tumors as advised by the reviewers, We can utilize transgenic mice that express a T cell receptor (TCR) specific for the Tyrosinase-Related Protein 1 (Trp-1).

We can measure antigen presentation by DCs to T cells in the presence and absence of physiological concentrations of NO from a nitric oxide donor SNAP or co-culture with NO producing MDSCs using activation and subsequent proliferation of T cells as the experimental observable. Specifically, CD4+ T cells can be stimulated in vitro with Trp-1 peptide-pulsed spleen-derived murine DCs that have been exposed to an NO donor (SNAP), MDSCs±iNOS inhibitor, melanoma in tumor bearing mouse (±anti-PD-1 antibody IP biweekly) or control reagent and then analyzed for their capacity to proliferate in a [³H]-thymidine incorporation assay and ability to bind Trp-1 specific tetramers. For controls We can use DCs and MDSCs from commercially available knockout mice that are deficient in the nitric oxide synthase (B6.129P2-Nos2^{tm1Lau}/J) or STAT1 (B6.129S(Cg)-Stat1^{tm1Dlv}/J). Additional controls can consist of DCs, CD4+ T cells, and MDSCs incubated with either the CD3 antibody or LPS (for general
inflammation). The DCs and T cells utilized in these experiments can be analyzed for nitration of STAT1, NFkB, LCK-1, as well as MHC, TCR and guanylate cyclase via immunoprecipitation of tyrosine nitrated proteins followed by immunoblot.
   b) the iNOS Inhibitors Increase Responsiveness to Anti-PD1 Therapy and Restore Antigen Presentation

NCX-4016 is a nitroaspirin and inhibits the nitric oxide synthase via a negative feedback mechanism. These compounds are available for clinical trial use, and have a toxicity profile that is favorable for evaluation in patients with advanced malignancies. I predict nitroaspirin compounds in combination with anti-PD-1 therapy can rescue the ability to stimulate CD4⁺ T cells and decrease tumor growth. The data demonstrate that the combination anti-PD-1 and NCX-4016 therapy is more efficacious than either treatment alone (FIG. 3) and iNOS expression is decreased with iNOS inhibitor in-vivo. We can use nitroaspirin alone and along with anti-PD-1 in therapeutically achievable doses in the murine B16 melanoma model to measure tumor growth curves and obtain specimens for analyses. To demonstrate restoration of antigen presentation in vivo, experiments can be performed by injecting C57BL/6 mice with

B16 melanoma cells and treating the mice with nitroaspirin alone or with anti-PD-1. On day 14 the mice can receive 5*10⁶ CD4⁺ Trp-1 T cells. After 48 hours, tumors can be harvested and measurement of Trp-1 CD4 T cell specific tetramers can be performed using flow cytometry.

c) Changes in the Levels of iNOS Expression, Nitration Status of Proteins and Immune Cell Subsets Containing NO Predict Response to iNOS Inhibitors and Anti-PD-1 Treatment

iNOS expression can be measured via IHC. Nitration of STAT1 in DCs derived from murine models treated with anti-PD-1±NCX-4016 can be confirmed by immunoprecipitation of DC protein lysates for nitrotyrosine followed by immunoblot with an antibody specific for STAT1 (FIG. 2D). Flow cytometric measurement of IFN-induced (α+γ) phospho-STAT1 in NO-treated DCs can confirm the inhibitory effects of nitration on Jak-STAT signal transduction. Nitric oxide producing MDSCs (CD11b⁺, Gr1⁺, along with monocytic and granulocytic subsets) in the peripheral blood and tumor microenvironment can be stained in an analogous fashion to the human samples. Mass spectrometry can also be utilized to identify the nitrated proteins within the melanoma cell, DC, T, other myeloid cell lysates after immunoprecipitation with anti-nitrotyrosine antibodies as performed in experiments for the human samples.

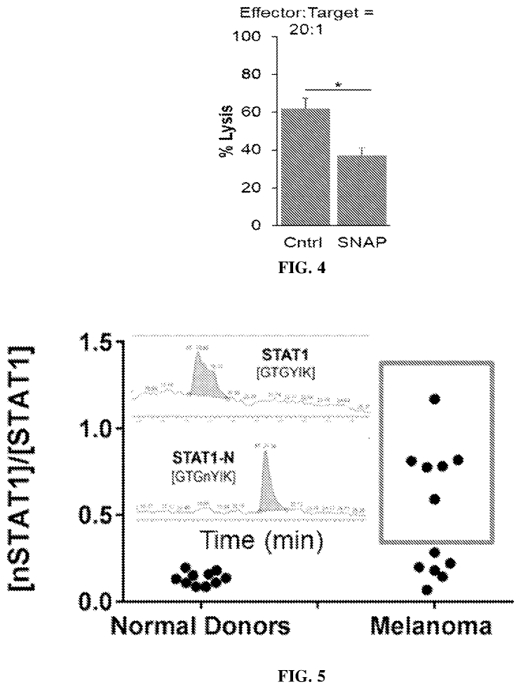

As antitumor responses are dependent on both CD4⁺ and CD8⁺ T cells, cross presentation between DC and CD8⁺ T cells can be analyzed utilizing a complementary murine model whose TCR recognize the gp100 peptide (aa 25-33) in the context of class I MHC (PMEL model) (FIG. 4). CD8⁺ T cells can be stimulated in vitro with peptide-pulsed spleen-derived murine DCs that have been exposed to an NO donor (SNAP), MDSCs (±nitroaspirin treatment), melanoma in tumor-bearing mouse (±anti-PD-1 300 μg IP twice weekly) or control reagent (PBS) and then analyzed for their capacity to proliferate in a [³H]-thymidine incorporation assay, to secrete immunomodulatory cytokines (IFN-γ, IL-2, and TNF-α), and lyse PMEL-expressing target cells in a standard 4 hour 51Cr release assay. Control DCs, CD8⁺ T cells or MDSCs can be subjected to the same conditions as for the Trp-1 CD4⁺ T cell model system. Collectively, these experiments elucidate how antigen presentation is inhibited by NO-dependent mechanisms and delineate new avenues for therapeutic development. Immunogenic YUMM melanoma cells (BRAF mutant/PTEN null) can be utilized to further confirm findings in the PMEL model.

### d) Modulation of Checkpoint Blockade by NO is Concentration-Dependent.

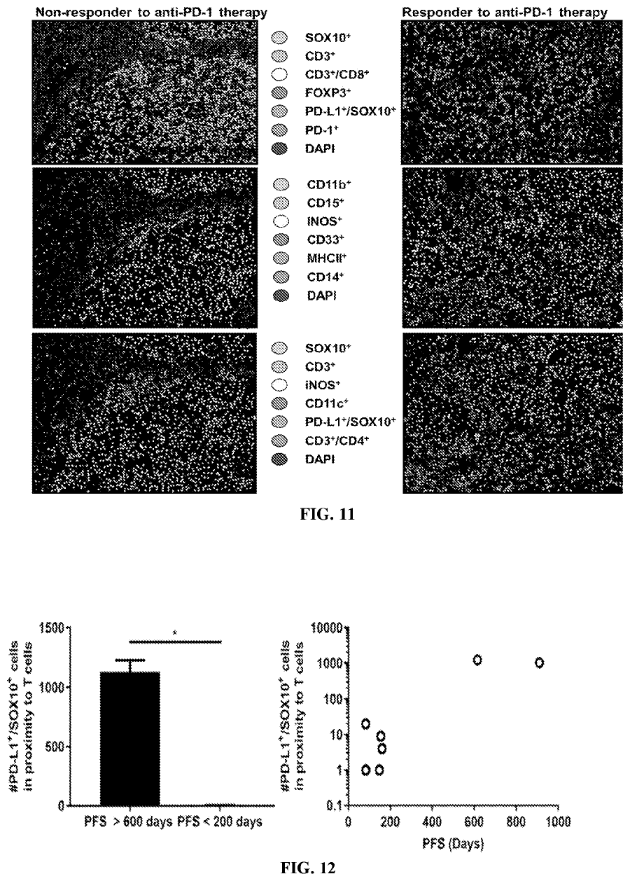

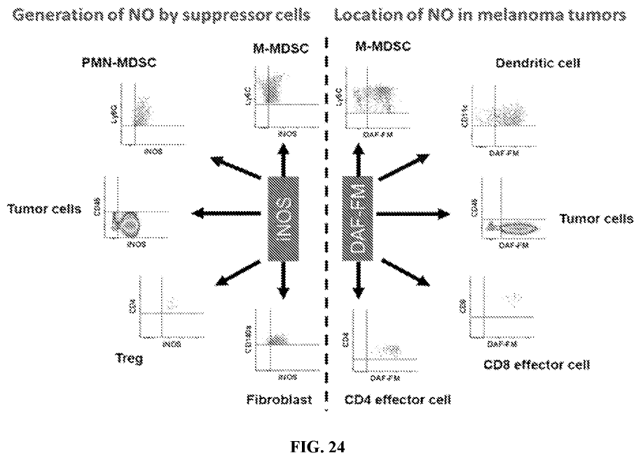

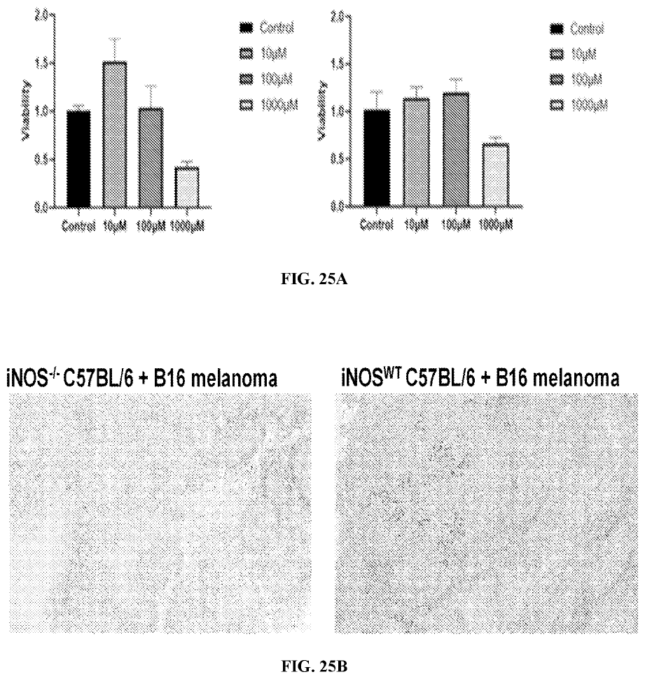

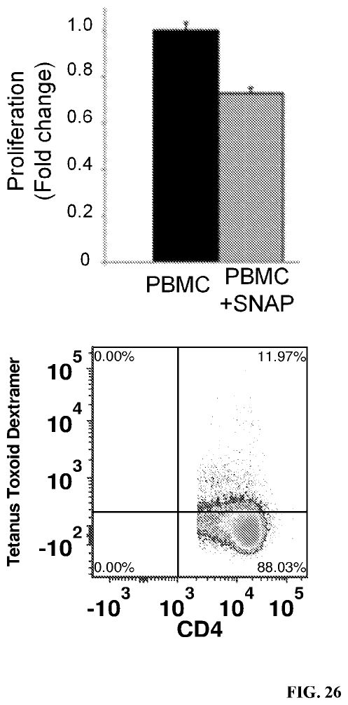

The mechanism of resistance to anti-PD-1 is dependent on the inability to turn off the production of nitric oxide after the initial NO burst kills melanoma cells. Murine models were used to show when and where NO production is turned on/off in the models and how this event leads to anti-PD-1 resistance. It was observed that NO was chronically elevated after the initial productive NO burst. Indeed, the data shows that smaller tumors have higher iNOS expression (FIGS. 25A and 25B) whereas tumors at endpoint have consistently low iNOS levels (FIG. 24). In this case, a mass spectrometry multiple reaction monitoring assay is performed on patient-derived specimens. In addition, MPATR analysis is utilized to assess the peripheral immune response in patients undergoing anti-PD-1 therapy. It is observed that the molecular subtype of melanoma creates no difference in the timing of the nitric oxide burst. BRAF WT, BRAF V600E and NRAS Q61 models have similar responses. This is based on that interferon led increase in iNOS expression and anti-PD-1 therapy mediates an interferon dependent effect whether or not patients respond to therapy (FIG. 11).

### Experimental Approach

Murine C57BL/6 Model with B16 Tumors

### Flow Cytometry:

The two flow panels for measuring the location of generation of NO (iNOS panel FIG. 24), vs Location of NO in melanoma tumors (DAF-FM panel) can be utilized 6 hours after anti-PD1 administration, day 3 and day 6. The MPATR analysis utilized to analyze the data to test the NO formation increases in immune suppressor cells as tumors increase in size.

### Statistical Considerations:

To correlate levels of NO producing MDSCs/other immune cells/nitration of proteins and tumor size in the presence or absence of anti-PD-1 therapy using the Spearman correlation coefficient. The levels of MDSCs were measured from peripheral blood of mice serially after treatment with anti-PD-1 and other checkpoint blockade agents alone or in combination to confirm the generality of the operative mechanism.

### Multiomics Transcriptomics and Proteomics:

Treat PBMC and cells lines with a titration of IFN-α/β/γ±NOSi (L-NAME) to determine the optimal concentration for elevation of NO (measured by DAF-FM, and Griess reagent). While NCX can have advantages in vivo, L-NAME has a direct inhibitory effect on iNOS and therefore is more suitable for these in vitro assays. Perform whole transcriptomics (HTG assay), phosphoproteomics and expression proteomics to examine the mesenchymal transition.

2 normal donor of PBMCs and 3 experimental replicates were utilized. 8 conditions were utilized: 1) Control 2) SNAP 3) IFN alpha 4) IFN beta 5) IFN gamma 6) IFN alpha+NOS inhibitor 7) IFN beta+NOS inhibitor 8) IFN gamma+NOS inhibitor. Human PBMCs can be treated with SNAP, IFN α/β/γ±NOS inhibitor and assayed on the HTG Diagnostics whole transcriptome array.

### Nitration in Cell Subsets:

T, DCs and MDSCs obtained from the normal donor PBMCs can be subjected to Mass Spectrometry Methods: Measure expression proteomics, phosphoproteomics of murine tumors exposed to anti-PD1 for 1, 2 and 3 days in addition to human PBMCs described above.

### Expression Proteomics:

Proteome analysis is performed by LC-MS/MS using tandem mass tag (TMT) chemical labeling for relative quantification of protein expression in multiplexed samples to compare PBMCs from melanoma patients with different responses to anti-PD-1 therapy. This approach reduces costs, improves precision, and gives comparable biological content to label-free LC-MS/MS. Peptide sequences detected in LC-MS/MS are identified by searching against species-specific UniProt database entries using Andromeda and quantified using MaxQuant. The resulting output can be viewed at the protein or peptide level; in both cases, the protein name or peptide sequence is provided in a table with the evidence for identification and the quantitative data for protein or peptide expression in each sample. Targeted proteomics with LC-selected reaction monitoring mass spectrometry (SRM) can also be used to translate these findings into assays that can be applied to evaluate protein expression in other model systems or panels of human tumors.

Global Profiling of Protein Phosphorylation: Proteins from cell lysates or homogenates are denatured, reduced, alkylated, and digested with trypsin. After buffer exchange, tyrosine-phosphorylated peptides are enriched using an anti-phosphotyrosine antibody (Cell Signaling); using the flow through, the remaining peptides are fractionated with basic pH reversed phase liquid chromatography prior to serine/threonine phosphopeptide enrichment with immobilized metal affinity chromatography (IMAC Fe-NTA Magnetic Beads, Cell Signaling). Phosphopeptides (global with or pTyr without TMT labeling) are analyzed with LC-MS/MS, identified with Andromeda, and quantified by MaxQuant.

Measurement of nitration via immunoblot: The DCs and T cells utilized in these experiments were analyzed for nitration of STAT1, NF-kB, Actin, and PUS7 via immunoblot of nitrotyrosine immunoprecipitation of cell subsets. Nitration of STAT1 in DCs derived from murine models treated with anti-PD-1±NCX-4016 were confirmed by immunoprecipitation of DC protein lysates for nitrotyrosine followed by immunoblot with an antibody specific for STAT1 (FIG. 2D) in addition to cytoskeletal elements such as actin. Flow cytometric measurement of IFN-induced (α+γ) phospho-STAT1 in NO-treated DCs can confirm the inhibitory effects of nitration on Jak-STAT signal transduction. The nitration of Actin and NF-kB is associated with disrupted cytoskeletal rearrangements needed for effector function subsequent to antigen presentation.

Functional Assays to determine localization of nitrated proteins and rearrangement of cytoskeleton. The ImageStream technology is utilized to follow signaling molecules (e.g. NF-kB inside T cells after nitration) upon stimulation with IFNs. These proteins cannot migrate to the nucleus and activate downstream effector elements.

### Example 2: Nitration of Specific Proteins in the IFN Response Pathways in Human Melanoma Specimens Results in a Decreased Response to Anti-PD-1 Therapy

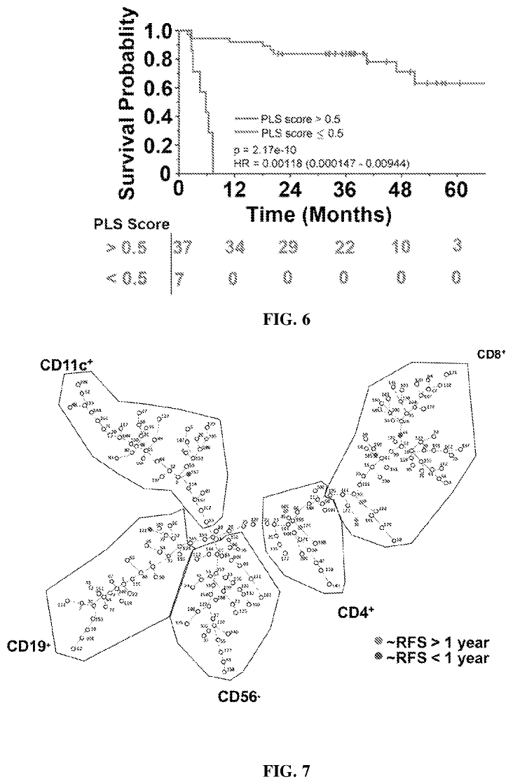

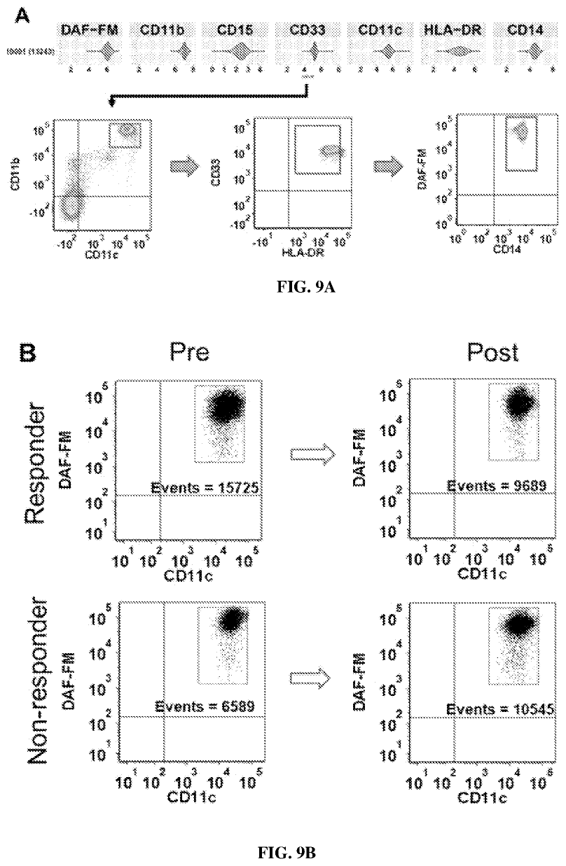

NO inhibits antigen presentation in melanoma and this effect can be abrogated by iNOS inhibitors such as NCX-4016. Herein we identify which patients have a very high likelihood of anti-PD-1 failure as a single agent, making these types of patients eligible in the future to block NO dependent processes. Clinically useful assays that can identify nitration events in peripheral blood must be developed. I developed a multiple reaction monitoring mass spectrometry method on the Thermofisher Quantiva system that can identify [nitrated STAT1]/[STAT1] from the PBMCs of melanoma patients. When the [nSTAT1]/[STAT1] ratio is >0.2, then the ratio discriminates between normal donor controls and melanoma patients progressing on current treatment with a small variance in the nitration levels in normal donors (FIG. 5). To expand the list of proteins important for NO-dependent primary melanoma cells were lysed, the proteins were extracted and digested with trypsin. Anti-nitrotyrosine antibodies conjugated to magnetic beads were utilized to immunoprecipitate nitrated peptides, which were then identified nitrated proteins including STAT1, using liquid chromatography-tandem mass spectrometry peptide sequencing which included detection of nitration of STAT1. Furthermore, expression proteomics and targeted mRNA experiments (HTG immune oncology array), 19 and 25 FFPE samples collected prior to anti-PD1 therapy, respectively have illustrated antigen presentation proteins important for response to anti-PD1 (FIG. 1). Multiple other proteins can be nitrated (NFkB, TCR, MHC, LCK-1) and measurement of STAT1 represents the first step in measurement of the “nitrome” to determine who is resistant to anti-PD-1 therapy. Furthermore, NO is elevated in MDSCs and other immune cell subsets as also illustrated by immune phenotyping tool MPATR (FIGS. 6-10). We have devised a signature (FIGS. 6-8) that can be tested in a validation cohort of samples with known outcomes (from ECOG 1609) and studied the mechanisms of protein nitration and phosphorylation as they relate to response to ipilimumab- and interferon-based therapy.

Immunofluorescence stains on FFPE tissues demonstrate increased iNOS in non-responder vs increased immune infiltration in responders (e.g. PDL1⁺/SOX10⁺ cells in proximity to T cells (FIGS. 11-12). Spatial examination is important as although the total MHC staining appears to be increased, MHC class II⁺ cells are present at the periphery of the tumor and CD4⁺ T cells are in close proximity to CD11c⁺ antigen presentation cells only in responders. Nitration of proteins such as STAT1, measurement of NO containing immune cell subsets, and mRNA/proteomics patterns in PBMCs collected from melanoma patients are associated with a decreased response to anti-PD-1 therapy and correlated with changes in immune cell responses to interferon levels and nitric oxide produced by immune cell subsets. These experiments show that NO targeted therapy can reverse anti-PD-1 resistance. The goal is to study how specific members of the nitrome beyond STAT1 confer resistance to checkpoint blockade and to facilitate translation of the science into the clinic. Informatics tools can be developed to analyze omics data.

a) Validation of Nitration of PUS7 Nitration in Human PBMCs Collected from 35 Patients (Pre and Post Anti-PD-1) is Associated with Changes in Immune Cell Subsets.

Experimental Method: Developed a selective reaction monitoring mass spectrometry method on the Thermofisher Alts system that can identify [PUS7] from the PBMCs of melanoma patients as done for STAT1 (FIG. 5). Performed SRMs for NFkB, Actin, FAK1 and the proteins seen in the murine experiments, but these were not found in the peripheral PBMCs and are likely intrinsic to the tumor cells and not the immune cells. However, during the course of these investigations using an unlabeled approach PUS7 was found to have increased tyrosine nitration at position 568,574 in circulating immune cells in those patients who have increased PFS (log 2 ratio=−0.42; p=0.03). This is significant as PUS7 functions to modify RNA molecules for signaling and has recently been implicated in other tumor progression such as glioblastoma multiforme. An increased [PUS7-nitrotyrosine] can be associated with resistance to anti-PD-1 therapy. Unfractionated PBMC samples are utilized for the mass spectrometry experiments as they are routinely collected from patients and with [PUS7] having increased nitration. The SRM for PUS7 was developed as for STAT1, STAT3, NFκB, HDAC1, and Actin.

In addition to specific biochemical modifications from NO, a phenotypic signatures of immune cells is elucidated containing nitric oxide metabolites important for patient specific survival (FIG. 6). Measure 35 patients before the first and second dose of anti-PD1 to obtain PBMCs for both nitration of PUS7 and other proteins as described above and for measurement of immune cell phenotypes to validate that it is a different signature between anti-PD1 and anti-CTLA4. Given the preponderance of lymphoid populations and data (FIG. 2) lymphoid populations is measured first with respect to Nitric oxide.

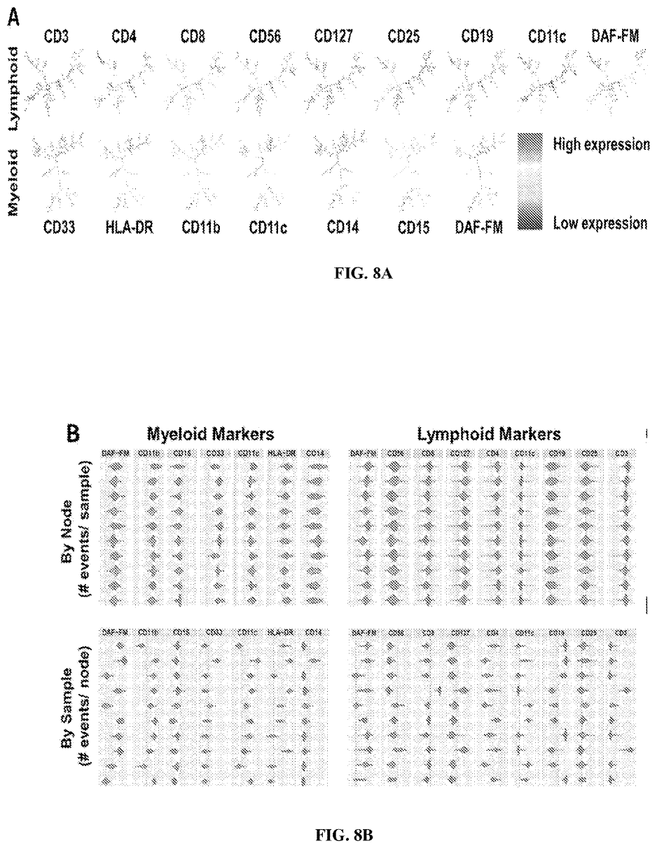

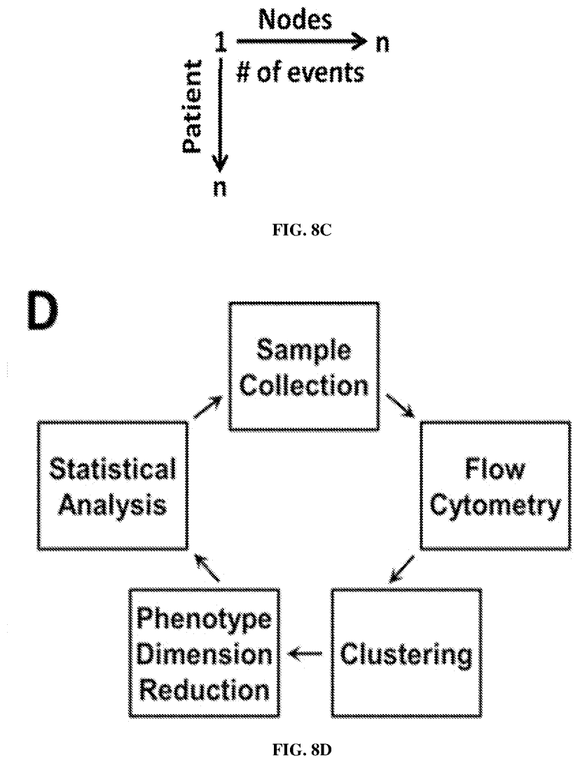

A optimized tool (MPATR) in our group (FIGS. 8 and 6) is utilized to measure clinically meaningful immune cell subsets from multidimensional flow cytometry experiments. NO levels were examined in immune cell subsets in PBMC samples. MDSCs within the peripheral blood of melanoma patients were phenotyped using a flow cytometric technique that employs fluorescently-labeled antibodies (with compatible fluorochromes) for CD33, CD11b, CD11c, HLA DR, CD14 (monocytic marker), and CD15 (granulocytic marker) in conjunction with 4-Amino-5-methylamino-2′,7′-difluorofluorescein diacetate (DAF-FM), a nitric oxide stain (FIG. 8). Other immune subsets and normal donor PBMCs were characterized in each experiment to determine their NO content as well. T cell subsets, DC subsets, NK cells and B cells within the peripheral blood of melanoma patients were phenotyped for NO production by flow cytometry using antibodies for CD3, CD4, CD8, CD11c, CD27, CD125, CD19, and CD56. The DAF-FM dye is provided in the diacetate form which enhances its passage through the cell plasma membrane and subsequent cleavage by intracellular esterases. DAF-FM then interacts with nitric oxide to give a fluorescent signal. Additional panels have also been developed that include immune suppressor/stimulatory markers such as CD69, TCRζ, PDL1, IFNγ, CD103, CTLA4 and FOXP3. Cells were stained, fixed in 1% para-formaldehyde, and analyzed on an BD Symphony or LSR II flow cytometer (100,000 live events) using standard gates, fluorescence minus one controls, isotype control antibodies and compensation beads to establish criteria for positive staining and compensation controls. The percentage of positively-staining cells and their mean fluorescence intensity (MFI) were calculated for cell populations of interest and the data can be processed using FCS Express software and the MPATR algorithm.

Statistical Method: To investigate the association between [PUS7] and response to anti-PD-1 therapy at 1 year in patients with unresectable stage III/IV metastatic disease, 34 patients were analyzed. The primary statistical endpoint is PFS (12 months) that is associated with a decreased [PUS7]. The consented patients had blood collected for use at 2 time points (immediately before treatment initiation on day 0 and prior to the 2nd dose of therapy, (2-4 weeks after the first dose of anti-PD-1 (generally, immediately before the patient receives their second dose of therapy). 40 patients were consented to account for attrition. The response definition is based on its clinical significance and utility by clinicians. The primary clinical outcomes of research collected are: 1) objective response (RECIST and immune-related response criteria) and complete response rate; 2) “clinical benefit rate” (R+PR+SD for at least 2 weeks). Additional outcomes collected are 1) Progression Free Survival time; 2) % progression free at 6 and 12 months; 3) duration of objective response from all patients and conduct exploratory analyses to see which endpoint(s) best correlate with immune dysfunction. The assay consistently identifies patients who will not gain at least one year of PFS with single agent-PD-1, then those patients are considered appropriate candidates for more aggressive treatment with the combination anti-CTLA-4/anti-PD-1 despite its higher toxicity and also appropriate for clinical trials of anti-PD-1 plus NO-modulating agents with a study design aimed at significantly increasing the percentage of patients who achieve 12 month PFS or better.

Sampling distribution of the [PUS7] ratio was examined, proper transformation was investigated and performed prior to parametric tests (e.g. log transformation) and visualized using histogram, dot plot and boxplot. The two-sample parametric t-test was performed first to investigate the association between the [PUS7] ratio transformed data and the treatment response. The [PUS7] ratio cut point was further investigated using ROC analyses with the cut point for the [PUS7] ratio maximizing the area under the curve. Other cut points were considered if clinically more meaningful. Discretized data was analyzed using the Fisher exact test.

Power/sample size justification: With the proposed 34 patients (with 24 patients progressing at 1 year and 10 progression free at 1 year), this achieved 80% power to detect a 1.7 fold change (in the original scale) with a significance level of 0.05. A 14% increase was detected from the mean [PUS7] ratio around 0.2 to 0.34 given the observed variability.

MPATR analysis: To test the signature for ipilimumab that stratifies the patients who received adjuvant pembrolizumab into those patients that have long or short PFS, respectively. A test to determine who responds poorly with adjuvant pembrolizumab/ipilimumab and therefore have an “enrichment score” for responders and non-responders. The new flow cytometry data sets were superimposed on the old phenotypic tree and a table of number of events in each node for each patient were assembled. The data analysis up to the point of debatching was performed in a similar way as described in the data for the lymphoid signature (FIG. 6). The 8 phenotypes were treated as being one signature (i.e. all 8 nodes contribute to the biomarker and can be treated as one variable). The model assumes that at least 16% of patients have PFS less than 1 year. This method is utilized to determine that there is a different phenotype needed for pembrolizumab/nivolumab treated patients to have increased relapse free survival.

b) Increased Nitration of the STAT1 vs. Other Proteins in PBMC Samples is Associated with Decreased Response to Anti-PD-1 Therapy

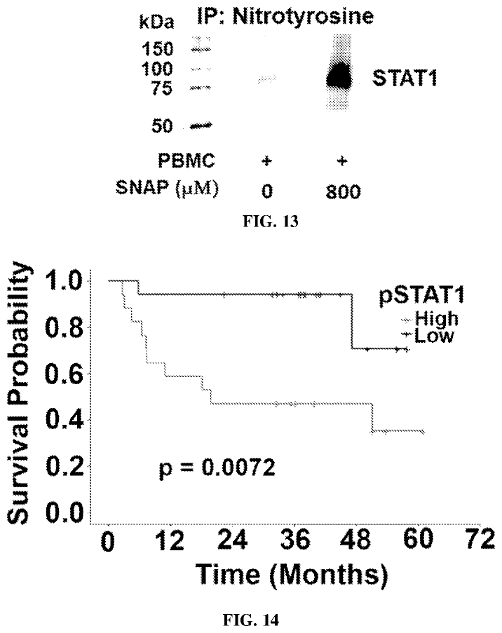

An increased [nSTAT1]/[STAT1] ratio can be associated with resistance to anti-PD-1 therapy. Unfractionated PBMC samples are utilized for the mass spectrometry experiments as they are routinely collected from patients and with the expectation of STAT1 having increased nitration (FIG. 4). A second aliquot of PBMCs from the patients can be stimulated in parallel with IFN and subsequent downstream activation determined via measurement of phosphorylated STAT1. Activation of STAT1 can be measured by intracellular staining for activated (i.e. phosphorylated) STAT1 using flow cytometry. IFN responsiveness decreases by ~30% in metastatic cancer patients. There is an inverse relationship between the levels of pSTAT1 and levels of nitrated STAT1. As a control, STAT1 nitration can be measured in normal donor PBMCs treated with SNAP via immunoblot (FIG. 13). As such, PBMCs from melanoma patients can be compared to normal donors. In parallel, We can perform a whole nitrotyrosine and phosphotyrosine proteomics with the proteomics core (each requiring only ~1 million cells using TMT multiplexing to increase detection across samples). Additional nitrated proteins can be quantified in the future via LC-MRM (Liquid Chromatography, multiple reaction monitoring) including the targets outlined in the introductory material (assay requires <200,000 cells); and can expand the list of which nitration events are important for response to anti-PD1 therapy in case STAT1 is not the only important target.

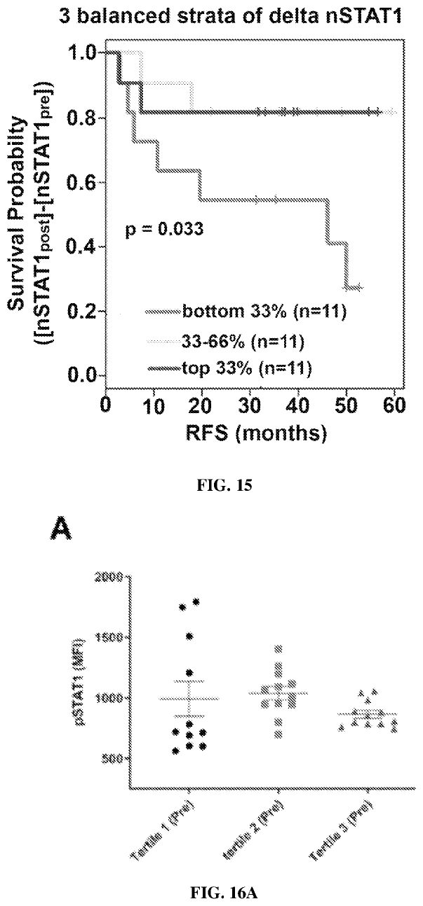

Analysis herein demonstrates that increased levels of pSTAT1 are associated with decreased RFS, indicating that the levels of pSTAT1 is in a narrow range for the interferon response to be effective in anti-CTLA-4 therapy (FIG. 14) to avoid “too much” stimulation of interferon pathways and their “exhaustion”. Furthermore, sustained interferon stimulation results in resistance to anti-PD-1 therapy. Changes in nitration are associated with increased RFS among this internal set of patients as measured in FIGS. 6-8 (FIG. 15). In these samples, sustained high levels of NO (bottom third of changes in nitration of Y701 of STAT1 (AnSTAT1) was associated with poor RFS. Therefore, using the samples obtained from ECOG 1609, we have developed a signature of response using NO phenotyping of immune cells.

c) Measure Levels of Nitric Oxide Produced by Immune Cell Subsets in PBMCs from Melanoma Patients Before and After Treatment with Anti-PD-1 and Correlate this with STAT1 Nitration, Phosphorylation and Changes in Immune Cell Responses to Interferon Levels

A newly optimized tool Multi-Dimensional Phenotyping Analysis Tool in R (MPATR) (FIG. 5) can be utilized to measure clinically meaningful immune cell subsets from flow cytometry experiments. In parallel studies, the ratio of nitrated STAT1 to non-nitrated STAT1 indicates poor outcomes to anti-PD-1 (FIG. 5). Importantly it appears that changes of STAT1 over time are important for anti-CTLA-4 therapy, whereas the baseline nitration levels are important for success/failure of anti-PD-1 therapy. This observation is key as CTLA-4 is traditionally felt to be important at the immune priming stage, whereas PD-1 is felt to be more important at the effector stage.

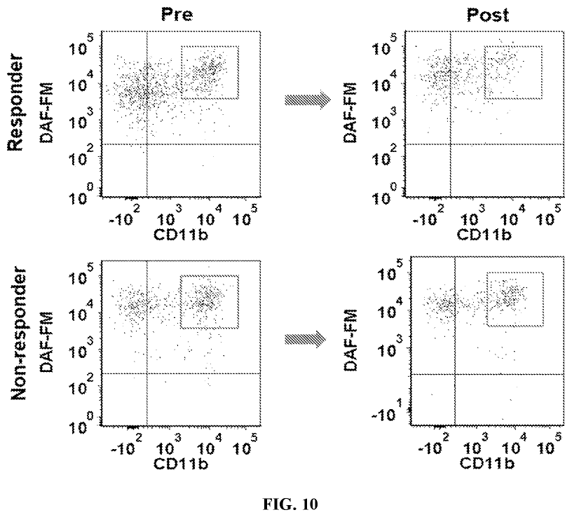

We can examine NO levels in immune cell subsets in PBMC samples. MDSCs within the peripheral blood of melanoma patients can be phenotyped using a flow cytometric technique that employs fluorescently-labeled antibodies (with compatible fluorochromes) for CD33, CD11b, CD11c, HLA-DR, CD14 (monocytic marker), and CD15 (granulocytic marker) in conjunction with 4-Amino-5-methylamino-2′,7′-difluorofluorescein diacetate (DAF-FM), a nitric oxide stain (FIG. 8). Although MDSCs are the major contributor of NO, we can characterize other immune subsets and normal donor PBMCs in each experiment to determine their NO content as well. T cell subsets, DC subsets, NK cells and B cells within the peripheral blood of melanoma patients can also be phenotyped for NO production by flow cytometry using antibodies for CD3, CD4, CD8, CD11c, CD27, CD125, CD19, and CD56. Additional panels have also been developed that include immune suppressor/stimulatory markers such as CD69, TCRζ, PDL1, IFNγ, CD103, CTLA4 and FOXP3. Cells can be stained, fixed in 1% para-formaldehyde, and analyzed on a BD Symphony or LSR II flow cytometer (100,000 live events) using standard gates, fluorescence minus one controls, isotype control antibodies and compensation beads (Invitrogen, Waltham, MA) to establish criteria for positive staining and compensation controls. The percentage of positively-staining cells and their mean fluorescence intensity (MFI) can be calculated for cell populations of interest and the data can be processed using FCS Express software (Glendale, CA) and MPATR. We can also expand the MPATR algorithm to the analysis of the tumor microenvironment (FIG. 10; CDP).

d) Bioinformatics/Biostatistics Methods for Analyzing Omics Based Data (mRNA Immune-Oncology Array, Proteomics) can Assess Involvement of NO and IFN Dependent Processes

mRNA and protein signatures can be measured from PBMC samples collected prior to and after anti-PD-1 therapy to see whether the NO pathway and interferon signatures are associated with response as seen in FFPE tissue specimens and suitable for a liquid biopsy for biomarker monitoring. PBMC samples can be run on an HTG EdgeSeq Processor (HTG Molecular Diagnostics, Tucson, AZ) using the HTG EdgeSeq mRNA immune-oncology assays (currently 1392 RNA transcripts of genes involved in the immune response to cancer including chemokines and checkpoint receptors) and expression/nitrotyrosine/phosphotyrosine proteomics can be run in the proteomics core. The mRNA panel can be normalized by dividing the abundance of each mRNA by the geometric mean of the housekeeping genes. Protein spectra, quantified using MaxQuant, can be normalized using IRON and filtered using the following cutoffs: log 2 ratio ≥log 2 (1.5-fold change), t test <0.05, and Hellinger distance >0.25. For proteomics data, an additional filter excluded rows mapping to bovine proteins or to reverse amino acid sequences (to define the false discovery rate). Pathway enrichment of differentially expressed genes was performed by applying Fisher exact tests to Molecular Signatures Database (MSigDB) gene lists. Prior to pathway enrichment of differentially expressed gene lists, the MSigDB gene lists were filtered to keep only those genes observed in their respective experimental proteomics, mRNA, or combined datasets to account for the immune-related bias of the targeted mRNA panel. Literature interaction networks of differentially expressed genes can also be generated with MetaCore (Clarivate Analytics). The output from the mRNA and proteomics data sets can be directly associated with the immune cell populations, levels of NO obtained from my MPATR analysis and levels of nitrated proteins involved in antigen presentation.

### Example 3: Assess how Anti-PD-1 Facilitates Nitration of Proteins and Mesenchymal Transition in Melanoma Cells

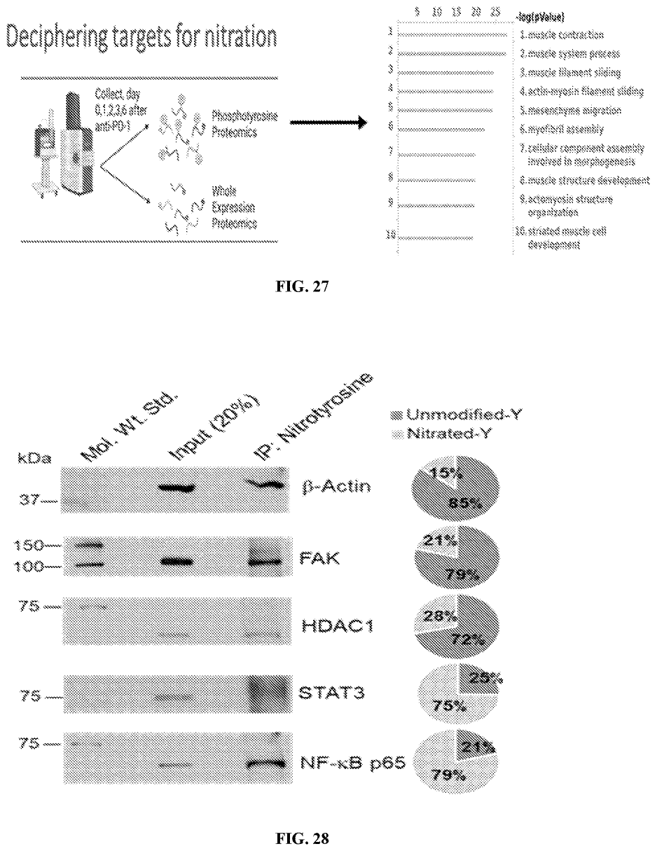

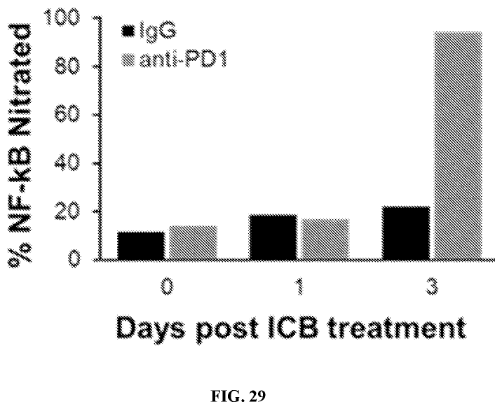

The phosphoproteome and expression proteomics were measured from B16-bearing C57BL6 mice treated with anti-PD-1 on day 0,1,2,3,6 post therapy (FIG. 27). Pathway enrichment of differentially expressed genes was performed by applying Fisher exact tests to Molecular Signatures Database (MSigDB) gene lists. Literature interaction networks of differentially expressed genes were generated with MetaCore (Clarivate Analytics). The phosphorylation of proteins were compared between day 3 and 6 post treatment (A [day 6 (anti-PD1-IgG)-day 3 (anti-PD-1-IgG)] when the melanomas start to grow more rapidly in the murine model and demonstrated processes involved in mesenchymal transition for the top 10 gene ontology terms (FIG. 27). To rule out the increase recruitment of fibroblasts flow cytometry panels were developed to include CD140a (FIG. 24) but morphophologically does not appear to be the case from H&E staining.

Once we had this list of proteins, the proteins were immunoprecipitated in B16 tumor samples with anti-nitrotyrosine antibody (FIG. 28) and demonstrated NF-κB, STAT3, FAK1, Actin, and HDAC1 are nitrated. It has been demonstrated that IFN signaling have a role in NO production via NF-κB/STAT1 pathways in patients who are being treated with anti-PD1 (FIG. 1, 26). This is consistent with the literature demonstrating that IFNs cause EMT transition in tumors. This data ascertains that generation of excess IFN support the mesenchymal transition of melanoma cells via stimulation of NO dependent pathways.

a) IFN-Mediated NO Generation Transition Melanoma Cells to be More Mesenchymal. SNAP and IFN Treatment of Melanoma Cells Drive Mesenchymal Transition

Experimental design: Treated BRAF WT and BRAF V600E melanoma cells lines with a titration of IFN-α/β/γ±NOSi (L-NAME) to determine the optimal concentration for elevation of NO (measured by DAF-FM, and Griess reagent). Used genetic tools to develop stable cell lines utilizing our genomics core to knock out iNOS or IFN-α/β/γ to determine which IFN is paired with nitric oxide production in mediating this effect. Specifically, the cell lines were treated with IFNs or a general inflammatory agent such as LPS and measure NOS activity via flow cytometry. L-NAME has a direct inhibitory effect on iNOS and therefore used for in vitro assays. Performed whole transcriptomics (HTG assay), phosphoproteomics and expression proteomics to confirm the interplay of iNOS with IFN and to examine the upregulated of genes and proteins involved in mesenchymal transition.

To conduct the multiomics experiments, utilized BRAF WT and V600E mutant cell lines with 8 conditions: 1) Control 2) SNAP 3) IFN alpha 4) IFN beta 5) IFN gamma 6) IFN alpha+NOS inhibitor 7) IFN beta+NOS inhibitor 8) IFN gamma+NOS inhibitor

We can also measure E-Cadherin and vimentin via immunoblot. To measure mobility of the cells traditional commercially available transwell migration assays were performed using serum starved cells. It was expected to see a 30% decrease in migration. In addition, performed nitrotyrosine immunoprecipitations followed by immunoblot to demonstrate whether NF-kB, STAT3, FAK1, are nitrated in the melanoma cell lines. In addition, to examine the cytoskeleton alterations, utilized standard immunofluorescence and live cell immunofluorescence microscopy of the actin cytoskeleton and molecules such as FAK1 (Focal Adhesion Kinase 1) to visualize morphology of the whole cell and nucleus along with rearrangement of cytoskeleton components in response to nitration.

Analysis: Whole array-based transcriptomics and the combination of this data with proteomics data were performed using informatics techniques. There was an approximately 30% decrease in migration. With ten replicate melanoma cell lines co-cultures with an NO donor (SNAP or IFN), there was 85% power to detect a 30% decrease (0.7 fold change) in proliferation compared to the control using a two sample t-test and assuming a 20% dispersion (CV=20%) of the data. An alpha level of 0.025 (0.05/2) for each end point is used to adjust for multiplicity. This effect was abrogated in the presence of the NOSi.

b) to Investigate how In Vivo Treatment of NOS Inhibitor with Anti-PD1 Abrogates Mesenchymal Transition and Blunts Melanoma Growth. Combinatorial Treatment of Mouse B16 Tumors with NOS Inhibitors (NCX, LNAME) and Anti-PD1 was Performed

Experimental design: A) Treated the B16D5 tumors as described above with anti-PD1±iNOS inhibitors (NCX and L-NAME). Collected the tumors to perform nitrotyrosine IP of targets NF-kB, STAT3, FAK1, and HDAC1. Performed immunoblots of E-cadherin and vimentin to demonstrate evidence that there is mesenchymal transition with anti-PD-1 treatment. Investigated the dependence of mesenchymal transition on iNOS and interferon dependent pathways. Used an iNOS KO line (B6.129P2-Nos2^{tm1Lau}/J; Jackson Lab) and WT littermates to develop B16 tumors with WT and iNOS-deficient B16 cells. The KO B16 cell lines were created. Tested that iNOS KO melanoma cells have increased efficacy with anti-PD-1 and decreased nitration of proteins seen in FIG. 28 to dissect the intra/extra tumoral role of iNOS facilitating protein nitration to initiate mesenchymal transition. Conditions included: anti-PD-1±NOSi, controls: IgG and vehicle control. Immunoblots as detailed in A) performed to demonstrate decreased nitration of proteins and decreased mesenchymal transition in the absence of iNOS in the tumor compartment. Performed whole expression proteomics and phosphoproteomics on these tumors at similar time points as described above in the tumors treated with anti-PD1±NOSi (controls: IgG and vehicle controls). Utilized genetics resources (Nos2^{tm2a(EUCOMM)Wtsi}; MGI) to create a conditional allele of NOS2 gene. The use of inducible-Cre driver provided the tool to study the cell and time-dependent role of iNOS in antigen presentation and mesenchymal transition.

Statistical Analysis: With ten replicate experiments with melanoma tumors±iNOS inhibitors, there is 85% power to detect a 30% decrease (0.7 fold change) in nitration of the proteins in the untreated samples treated with NOSi using a two sample t-test and assuming a 20% dispersion (CV=20%) of the data. An alpha level of 0.025 (0.05/2) for each end point is used to adjust for multiplicity. Given the statistics for nitration of proteins and the fact that the growth curves showed statistical significance with a lot of 5 mice per group, we are expecting 10 mice to confirm the statistical significance of the growth curves shown in FIG. 3.

### Example 4: Dichotomous Nitric Oxide-Dependent Post-Translational Modifications of STAT are Associated with Ipilimumab Benefit in Melanoma

Nitric oxide is typically thought of as an inhibitory molecule is cancer. In our studies nitric oxide (NO) was increased in immune effector cells among patients with longer RFS after adjuvant ipilimumab, whereas NO increased in immune suppressor cells among patients with shorter RFS. Herein, we utilize samples derived from the same patients to measure post-translational modifications of STAT1 (nitration-nSTAT1 and phosphorylation-pSTAT1) important for regulating its activity via flow cytometry and mass spectrometry approaches. Ipilimumab-treated patients with high nSTAT1 levels before and after therapy in PBMCs experienced decreased RFS but the change nSTAT1 levels before and after ipilimumab therapy was associated with longer RFS. This study reveals a dichotomous role for nitric oxide in melanoma and can lead to therapeutic implications.

Ipilimumab (anti-CTLA-4), although FDA-approved for stage III/IV melanoma adjuvant treatment, is not used clinically in first-line therapy given superior relapse-free survival (RFS)/toxicity benefits of anti-PD-1 therapy. However, it is important to understand anti-CTLA-4's mechanistic contribution to combination anti-PD-1/CTLA-4 therapy and investigate anti-CTLA-4 therapy for BRAF-wild type melanoma cases re-resected after previous adjuvant anti-PD-1 therapy. Our group published that nitric oxide (NO) was increased in immune effector cells among patients with longer RFS after adjuvant ipilimumab, whereas NO increased in immune suppressor cells among patients with shorter RFS. Herein, we measured post-translational modifications of STAT1 (nitration-nSTAT1 and phosphorylation-pSTAT1) important for regulating its activity via flow cytometry and mass spectrometry approaches. PBMCs were analyzed from 35 patients undergoing adjuvant ipilimumab treatment. Shorter RFS was associated with higher pSTAT1 levels before (P=0.007) and after (P=0.036) ipilimumab. Ipilimumab-treated patients with high nSTAT1 levels before and after therapy in PBMCs experienced decreased RFS but the change in nSTAT1 levels before and after ipilimumab therapy was associated with longer RFS (P=0.01). Measurement of posttranslational modifications in STAT1 can distinguish patients with prolonged RFS to ipilimumab and provide mechanistic insight into response to ipilimumab combination regimens.

The FDA-registered immune-based therapeutics for resected stage III/IV melanoma include anti-PD-1, anti-CTLA-4, and interferon-based therapies. Several clinical trials completed in the past few years have guided clinical care. The Eastern Cooperative Oncology Group (ECOG)-1609 (stage IIIB-IV melanoma) trial reported increased relapse-free survival (RFS) with adjuvant anti-CTLA-4 therapies versus high-dose interferon-based therapies (interferon-alfa-2b). The Checkmate 238 trial compared nivolumab to ipilimumab for adjuvant treatment of stage IIIB through IV melanoma and demonstrated a 70.5% 12-month RFS rate among the nivolumab group compared to 60.8% among the ipilimumab group, along with significantly decreased toxicity. A recently published trial by the Southwest Oncology Group (SWOG) demonstrated increased efficacy of pembrolizumab for stage III melanoma compared to interferon-α or ipilimumab, which were the standards of care at the time.

Based on these data, the side-effect profile, and the perceived benefit of anti-PD-1-based agents, nivolumab or pembrolizumab are prescribed preferentially in the adjuvant setting for melanoma. Ipilimumab today is still considered clinically in the setting of a BRAF wild-type patient who was re-resected after previous adjuvant anti-PD-1 therapy. Furthermore, the RFS for anti-CTLA-4 regimens at 1 year in various trials ranges from 60% to 70%. One of the early trials of adjuvant ipilimumab activity investigated ipilimumab in combination with a peptide vaccine. In 2020, a follow-up study using samples from that trial demonstrated that nitric oxide (NO) was increased in immune effector cells involved in antigen presentation in patients with longer RFS following ipilimumab, whereas NO was increased in immune suppressor cells among patients with shorter RFS. Our group reviewed the effects of NO in melanoma, and there are multiple instances where NO mediates immune suppression. In contrast, in other circumstances, NO can promote the ability of immune effector cells to kill melanoma tumors. These two papers suggested a dichotomous effect of NO where immune effector cells can utilize NO to kill melanoma cells, whereas immune suppressor cells can use NO to limit the immune effector cells' ability to kill melanoma cells by post-translational modification of proteins. This study focuses on the role of nitration of STAT1.

It is known that the presentation of antigens by dendritic cells to T cells is defective in melanoma. Interferon signaling associated with STAT1 phosphorylation at tyrosine 701 by JAKs leads to dimerization of STAT1 and nuclear translocation to regulate interferon-stimulated genes and leads to appropriate immune surveillance of cancer. The phosphorylation of STAT1 is measured via multiparametric flow cytometry in peripheral blood mononuclear cells (PBMCs). In contrast to signal transduction activation by phosphorylation, nitration of STAT1 in myeloid-derived suppressor cells can cause immune inhibition.

Our recently published study of patients receiving ipilimumab also demonstrated increased levels of phosphorylated STAT1 (pSTAT1) among patients who had RFS of more than 12 months. Given the overlap in pSTAT1 levels among patients whose disease relapsed vs those whose disease did not, pSTAT1 levels were analyzed herein using Cox regression and Kaplan-Meier analyses. Resting pSTAT1 levels at day 0 and day 150 of adjuvant ipilimumab treatment were compared, in addition to pSTAT1 levels of these same PBMCs after they were stimulated with exogenous interferon-α, to illustrate the importance of intermediate levels of interferon stimulation.

The tyrosine located at the 701 amino acid (Y701) of STAT1 protein can also be nitrated, and increased nitration of Y701 in both murine splenocytes and human PBMCs is associated with tumor progression. To investigate STAT1 nitration, our group used selective reaction monitoring (SRM) for the quantitative measurement of STAT1 nitration at Y701, which is the STAT1 phosphorylation site that initiates nuclear translocation. The effect of STAT1 nitration in PBMC from patients treated with adjuvant ipilimumab on RFS was examined. The results revealed the importance of time-dependent changes of tyrosine nitration in PBMCs obtained from patients with melanoma who were undergoing anti-CTLA-4 therapy and experienced long RFS rates. In addition, the data demonstrated the importance of intermediate pSTAT1 levels in PBMCs prior to ipilimumab therapy.

### a) Materials and Methods

Patient samples. Archived PBMC samples were obtained from patients who previously participated in a phase II trial of adjuvant ipilimumab with a peptide vaccine, and the treatment regimen has been described. Peripheral blood leukocytes were collected before initiation of ipilimumab treatment and at approximately 150 days after the first treatment. PBMCs were available and were de-identified before inclusion in our current study.

Flow cytometry and mass spectrometry analyses. Flow cytometric analysis of pSTAT1 and liquid chromatography-tandem mass spectrometry (LC-MS-MS) analysis of nitrated STAT1 (nSTAT1) were conducted on all samples. For flow cytometry studies, frozen PBMCs were thawed at 37° C., washed with culturing media, and allowed to rest overnight in complete media at 5% CO₂ at 37° C. PBMCs were stimulated with interferon-α (Miltenyi Biotec, Cambridge, MA) at 0, 10² U/mL, and 10⁴ U/mL and incubated for 15 minutes in media.

The live/dead marker Zombie NIR (BioLegend, San Diego, CA) was used before permeabilization to distinguish live cells. The samples were permeabilized using the FIX & PERM Cell Permeabilization Kit with methanol modification (Fisher Scientific, Hampton, MA) and fixed at −20° C. for a minimum of 2 hours. pSTAT1 was detected by a pSTAT1 antibody (AF488; BD Biosciences, San Jose, CA). Flow cytometry data were collected either on Canto or LSRII flow cytometers (BD Biosciences, San Jose, CA), and data were analyzed in FCS Express (De Novo Software, Pasadena, CA). Measurement of patient-derived PBMCs for nSTAT1 and native STAT1 via LC-MS-MS SRM experiments was performed.

Statistical analyses. We conducted statistical analyses to determine whether there is a relationship between levels of pSTAT1, nSTAT1, and RFS. Survival outcome was summarized using Kaplan-Meier method and log-rank test was performed to evaluate the association of survival outcome with pSTAT1 and nSTAT1 levels dichotomized by the median-split method. The prognostic effect (hazard ratio and 95% confidence interval) of continuous pSTAT1 and nSTAT1 levels, with or without interferon-α stimulation, on RFS was evaluated using a Cox's proportional hazards regression model. Levene's F-test for the homogeneity of variance was used to compare variance of pSTAT1 level among patients with different RFS rates. Patients were also stratified using the median-split method for a survival comparison analysis of [nSTAT1]ₚₒₛₜ-[nSTAT1]ₚᵣₑ with ipilimumab treatment. The statistical analyses were completed using SAS 9.4, R Studio 3.5.3 (http://www.r-project.org), and GraphPad Prism 7.05. A two-sided P value of <0.05 was considered statistically significant. Treatment outcome is reported at the end of the follow up period for the parent trial (5 years).

TABLE 1
Patient characteristics (n = 35).

| | | |
|---|---|---|
| Characteristic | n | Percentage |

Gender

| | | |
|---|---|---|
| Male | 22 | 63 |
| Female | 13 | 37 |

Melanoma stage

| | | |
|---|---|---|
| IIIC | 16 | 46 |
| IV | 19 | 54 |

Ipilimumab dose

| | | |
|---|---|---|
| 3 mg/kg | 6 | 17 |
| 10 mg/kg | 29 | 83 |

Treatment outcome

| | | |
|---|---|---|
| NED | 27 | 77 |
| AWD | 2 | 6 |
| DOD | 6 | 17 |

Age

| | | |
|---|---|---|
| Median | 58 years | — |
| Range | 21-78 years | |

Abbreviations: AWD, alive with disease; DOD, died of disease; NED, no evidence of disease.

### b) Results

Patient demographics. Pre- and post-therapy PBMCs were available from 35 patients with resected stages IIIC/IV melanoma. The median patient age was 58 years (range, 21-78 years), and 63% of patients were male. Sixteen (46%) patients had stage III disease; 19 (54%) had stage IV disease. Six (17%) patients received ipilimumab at 3 mg/kg, and the remaining 29 (83%) received 10 mg/kg. The characteristics of the patient subset used in this study are summarized in Table 1. There was no statistical significant differences in RFS between the group of patients who received 3 mg/kg vs those who received 10 mg/kg (Log rank P-value=0.96).

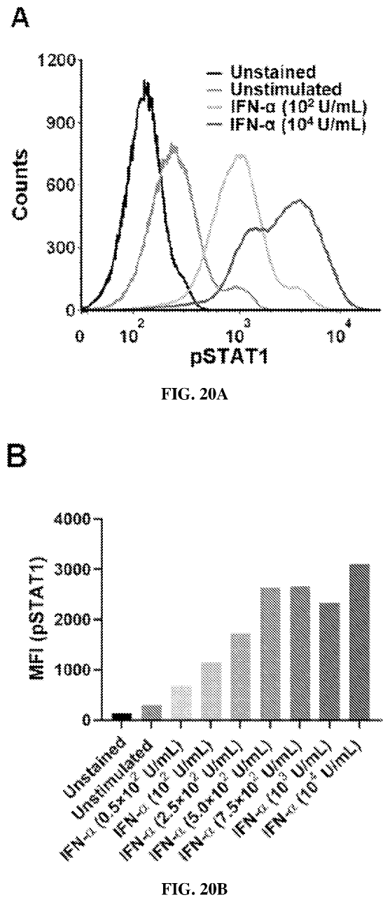

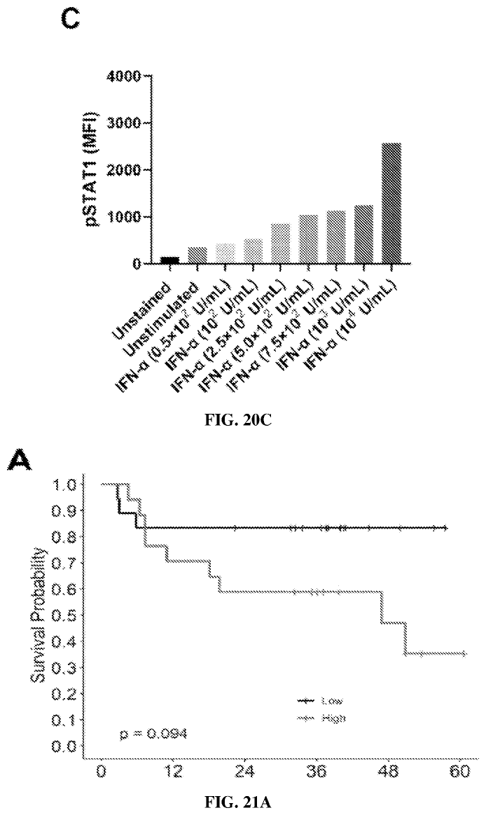

Type-I interferon treatment increased phosphorylation of STAT1. It is known that the maximum level of pSTAT1 achievable in PBMCs is different between patients, as described by Lensinski et al. First, we explored the interferon concentrations needed for stimulation and saturation of pSTAT1 formation in response to exogenous interferon-α treatment. Normal donor PBMCs were isolated, and we tracked the interferon dose-dependent increase in pSTAT1 levels. An interferon concentration-dependent increase in pSTAT1 levels was seen with increasing interferon levels (FIG. 20). A concentration of 500 U/ml of interferon-α showed near-maximum phosphorylation of STAT1 in PBMCs following 15 minutes of treatment, whereas another normal donor demonstrated increasing pSTAT1 levels throughout the 10⁴ U/mL maximal dose (FIG. 20). Interferon-α concentrations of 10² U/ml (mimicking subsaturated stimulation) and 10⁴ U/ml (representing maximum stimulation of JAK-STAT signaling) were subsequently used for ex vivo stimulation of patient-derived PBMCs.

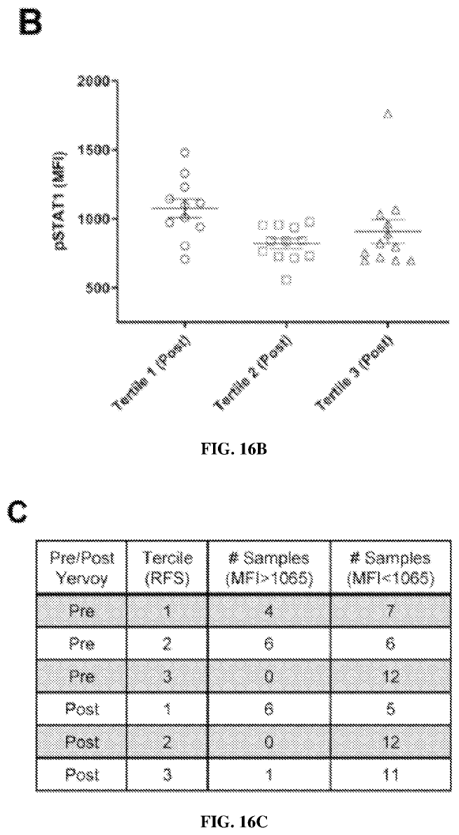

Phosphorylation of STAT1 displays a narrow distribution in samples with long RFS. The relationship of pSTAT1 levels with RFS was analyzed by dividing the patients into even terciles by RFS. The terciles were defined as follows: tercile 1, RFS<24 months; tercile 2, RFS between 24 and 40.3 months; tercile 3, RFS>40.3 months. As shown in FIG. 1, patients in tercile 1 (shortest RFS) had a large variation in the mean pSTAT1 level at baseline before ipilimumab treatment. Patients in tercile 2 (intermediate RFS) demonstrated a less variable distribution in comparison to tercile 1 (P=0.01, Levene's F-test). Interestingly, patients in tercile 3 (longest RFS) demonstrated a narrow distribution of pSTAT1 levels before ipilimumab treatment, and none of the samples displayed pSTAT1 Mean Fluorescence Intensity >1065 unlike tercile 1 (FIG. 16A). This trend of a smaller variance of pSTAT1 among patients with higher RFS rates was retained after ipilimumab administration but did not reach significance, except for comparing tercile 2 to tercile 3 (P=0.008, Levene's F-test; FIG. 16B). This data indicates the importance of intermediate pSTAT1 levels in PBMCs prior to ipilimumab therapy.

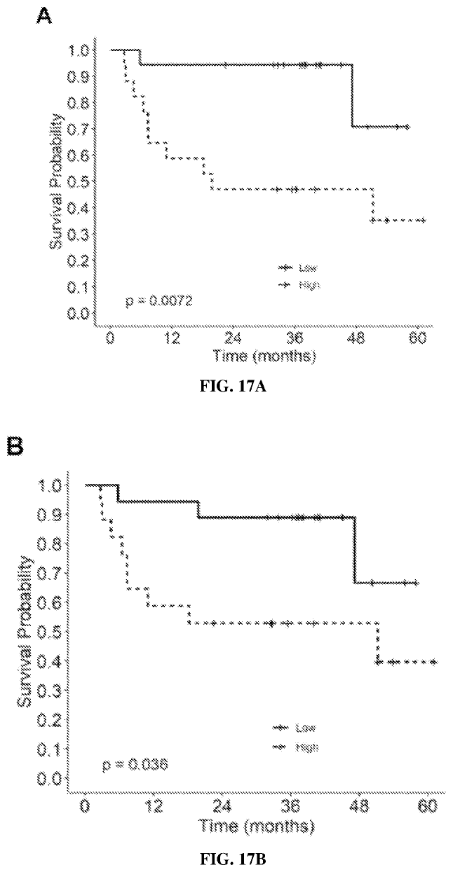

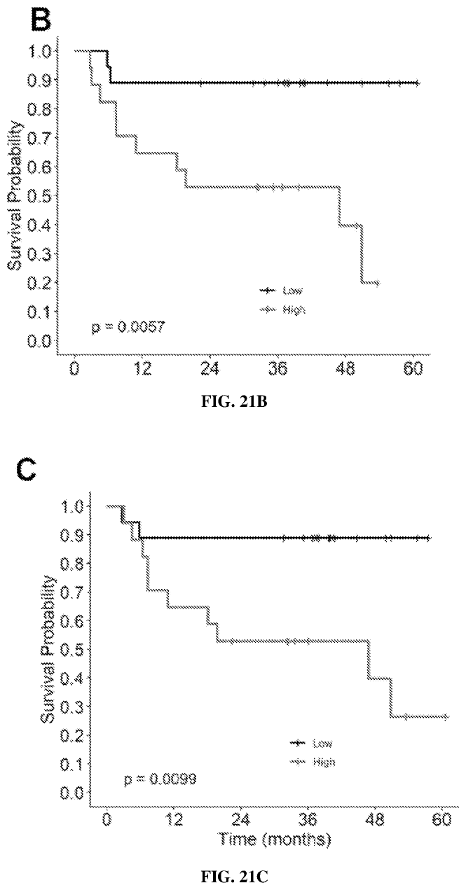

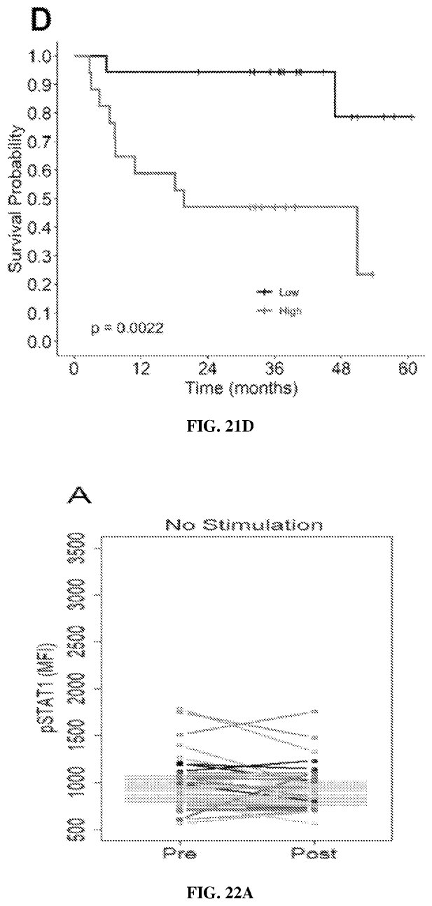

Higher levels of pSTAT1 were associated with low RFS in anti-CTLA-4 adjuvant settings. To examine the effect of steady-state phosphorylation of STAT1 on relapse-free survival in the setting of anti CTLA-4 adjuvant therapy, we stratified patients into 2 groups (above or below the median pSTAT1 level) and analyzed the relapse-free survival probabilities of each group before and after 150 days of ipilimumab administration. The Kaplan-Meier survival curve demonstrated worst survival in the group with higher pSTAT1 both before (P=0.007; FIG. 17A) and after (P=0.036; FIG. 17B) adjuvant ipilimumab treatment. Consistently, patient samples that demonstrated high pSTAT1 levels following stimulation with exogenous treatment of interferon-α (10² and 10⁴ U/ml) were among the worse survival groups (FIG. 21). This result indicates that increased pSTAT1 levels beyond a certain point do not lead to favorable outcomes following anti-CTLA-4 adjuvant therapy for melanoma.

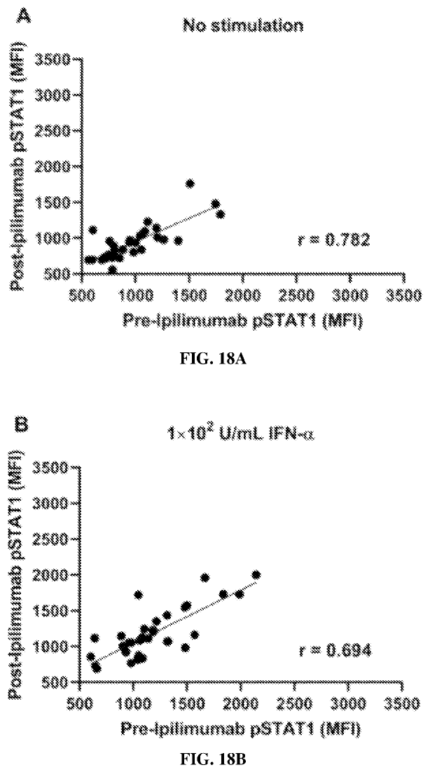

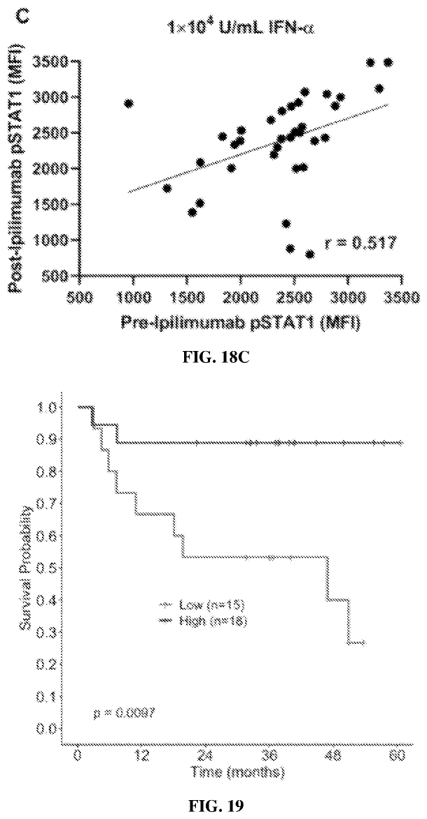

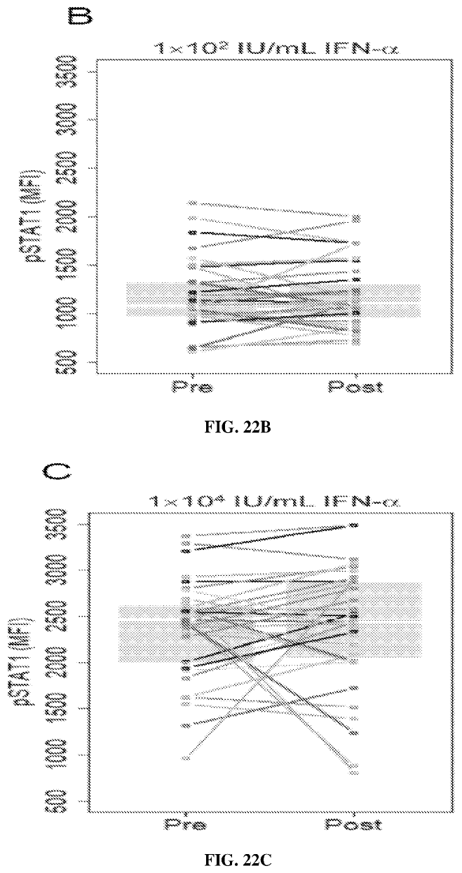

Stimulation with 10² U/ml of interferon-α demonstrated comparable pSTAT1 activation before and after ipilimumab treatment. We studied the effect of anti-CTLA-4 therapy on the capacity of PBMCs to stimulate STAT1 with 10² U/ml, and 10⁴ U/ml of interferon-α ex vivo. As shown in FIG. 18A, there is a linear relationship (r=0.78, P<0.001) between pSTAT1 expression on interferon-treated PBMC pre- and post-ipilimumab. There was a linear correlation between of PBMCs treated with intermediate doses of interferon-α (10² U/ml) obtained from patients pre- and post-ipilimumab treatment (r=0.69, P<0.001). This correlation was weakened (r=0.517, P=0.002) at a higher concentration of interferon-α (10⁴ U/ml) (FIG. 18B, C). Comparing the correlation coefficients demonstrated no difference between no stimulation and 10² U/mL of interferon-α (P=0.82), whereas a significant difference in the correlation coefficient was found when comparing no stimulation to 10⁴ U/mL of interferon-α (P=0.004) or comparing 10² U/mL of interferon-α to 10⁴ U/mL of interferon-α (P=0.002). Present data indicate that, in post ipilimumab therapy, PBMCs retain the pretreatment capacity to phosphorylate STAT1 in response to intermediate levels of interferon-α stimulation. However, with a higher dose of interferon-α, this capacity was compromised (FIG. 22). There were no statistically significant changes in pSTAT1 at 150 days post treatment between the 10 mg/kg and 3 mg/kg cohorts. Because nitration also occurs at position 701, in the next series of experiments, we investigated the nitration of STAT1 in PBMCs.

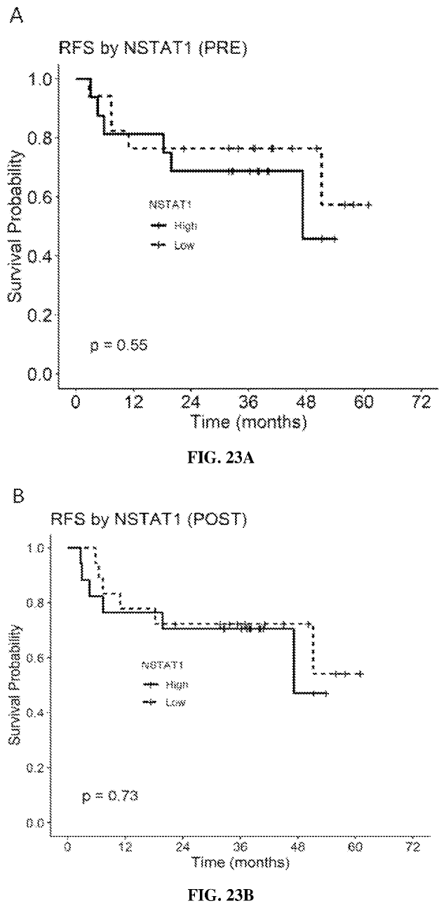

Increased [nSTAT1]ₚₒₛₜ-[nSTAT1]ₚᵣₑ in PBMC is associated with longer RFS. Given the above observations of pSTAT1 stimulation pre- and post-ipilimumab therapy, we then analyzed the nitration of Y701 before and after adjuvant therapy at the same timepoints as measured for pSTAT1. The magnitude of change in nSTAT1 levels pre- and post-ipilimumab therapy [nSTAT1]ₚₒₛₜ-[nSTAT1]ₚᵣₑ was measured via LC-MS-MS SRM techniques. Like the pSTAT1 analyses, patients were divided into 2 groups (above and below the median) based on the magnitude of change in nSTAT1 concentration. Unlike phosphorylation, nitration of STAT1 did not reveal a significant relationship with RFS either before or after anti-CTLA-4 administration (FIG. 23). As shown in FIG. 19, patients with a higher magnitude of increase following ipilimumab therapy showed improved relapse free survival probability, whereas patients with the lower magnitude of change in nSTAT1 had worse survival (P=0.01). These data also indicate that changes in nitration of STAT1 tyrosine 701 can influence JAK-STAT signaling after ipilimumab therapy.

### c) Discussion

A cohort of patients with stage IIIC/IV melanoma were analyzed to examine the relationship between STAT1 post translational modifications and RFS in response to anti-CTLA-4 adjuvant therapy. A narrow range of stimulation of interferon pathways, as measured by pSTAT1, is important for optimal response to anti-CTLA-4 therapy. Furthermore, increases in [nSTAT1]ₚₒₛₜ-[nSTAT1]ₚᵣₑ correspond to longer RFS among patients receiving adjuvant ipilimumab. The effects of STAT1 nitration on RFS are dependent on the changes of nitration in STAT1 Y701.

Nitration is a stable posttranslational modification. In addition, NO has a dichotomous role, being both immune stimulatory and inhibitory in PBMCs collected from patients receiving adjuvant ipilimumab therapy. Nitration of STAT1 blocks the phosphorylation of STAT1 and inhibits antigen presentation from dendritic cells to T cells. In other settings, NO can increase the killing of melanoma cells and is reviewed elsewhere. Given the dichotomous effects of NO and the ability of phosphorylated STAT1 to modulate interferon responses, this study measured the nitration of STAT1 in patients who underwent ipilimumab treatment. This is the first report to our knowledge of increased survival associated with the nitration of a protein (e.g., STAT1) in melanoma. Nitration inhibits the phosphorylation of STAT1 in vitro and in vivo. However, the phosphorylation of tyrosine is more labile than nitration, as indicated by the fact that nitration of tyrosine at position 701 results in less phosphorylation in murine models. Therefore, limiting the phosphorylation in patients past a certain point can prevent aberrant immune stimulation and subsequent immune suppression. Changes in the nitration status of STAT1 are associated with RFS, which supports the notion that modulation of the interferon response pathway is important for clinical response. Interferon-α is FDA-approved and extensively studied for use in melanoma adjuvant therapy, albeit with a high toxicity profile. Interferon-α exerts its molecular effects on melanoma in various ways (immunoregulatory, antiangiogenic, and proapoptotic). It promotes antitumor immunity by enhancing the function of both CD4 and CD8 T cells by positively effecting maturation, survival, and antigen presentation of dendritic cells. Indeed, high-dose interferon (HDI) adjuvant therapy of interferon-α (induction therapy consisted of 30 days of 20 MU/m² of intravenous interferon-α daily; maintenance consisted of 10 MU/m² given subcutaneously thrice weekly for one year) was the standard regimen that showed clinical benefits among patients with high-risk melanoma before using more efficacious checkpoint blockade agents.

In patients treated with HDI, patients who exhibited changes of pSTAT1 levels after induction around the median had increased RFS, whereas patients who exhibited no change or higher levels of change experienced worse survival rates. Likewise, in our cohort, prolonged RFS was noticed among patients with lower levels of pSTAT1 within a narrow range at baseline (day 0), and similar trends were observed at day 150. Both the pre- and post-therapy data demonstrated that patients with higher levels of pSTAT1 experienced shorter RFS. Likewise, it has been reported that a high dose of interferon-α does not show a more effective induction of interferon-stimulated genes than an intermediate dose.

In anti-PD-1 therapy for melanoma, enhanced interferon signaling before therapy also results in more favorable outcomes, but markedly elevated or sustained interferon signaling can result in poorer outcomes for patients. Nitration of STAT1 with adjuvant anti-CTLA4 therapy can modulate interferon signaling to promote longer RFS. These observations provide a foundation for developing future strategies aimed to optimize interferon signaling in all available adjuvant immunotherapy settings, and these concepts can be expanded to the metastatic setting. Indeed, a study analyzing dose-optimization in HDI settings showed clinical precedence of lowering the standard subcutaneous dose of interferon-α based on maximal activation (measurement of pSTAT1 levels) of the immune system. Phosphorylation of STAT1 was also utilized as the experimental readout in our study system given experience and importance.

In the current study, increased nitration after anti-CTLA-4 therapy is associated with prolonged RFS whereas in most cancer studies increased nitration is associated with immune suppression and disease progression at a single time point. Together with our results demonstrating the pro- and anti-tumor nature of nitric oxide, the results reported herein indicate that measurement of nitric oxide dependent events and modulation of interferon dependent pathways can distinguish patients who have prolonged RFS with anti-CTLA-4 antibodies compared to published responses to anti-PD1 based adjuvant checkpoint blockade in melanoma. This study indicates that nitration of proteins cany modulate immune checkpoint blockade such as ipilimumab and that it is feasible to measure these modifications from patient samples. In addition, it provides insights into the mechanism of response amenable to clinical/translational study of combination anti-PD-1/CTLA-4 therapy in that moderate interferon responsiveness is best for checkpoint blockade.

### REFERENCES

A. Garcia-Diaz, D. S. Shin, B. H. Moreno, J. Saco, H. Escuin-Ordinas, G. A. Rodriguez, J. M. Zaretsky, L. Sun, W. Hugo, X. Wang, G. Parisi, C. P. Saus, D. Y. Torrejon, T. G. Graeber, B. Comin-Anduix, S. Hu-Lieskovan, R. Damoiseaux, R. S. Lo, A. Ribas, Interferon Receptor Signaling Pathways Regulating PD-L1 and PD-L2 Expression, Cell Rep, 19 (2017) 1189-1201.
A. Liberzon, A. Subramanian, R. Pinchback, H. Thorvaldsdottir, P. Tamayo, J. P. Mesirov, Molecular signatures database (MSigDB) 3.0, Bioinformatics, 27 (2011) 1739-1740.
A. Liberzon, C. Birger, H. Thorvaldsdottir, M. Ghandi, J. P. Mesirov, P. Tamayo, The Molecular Signatures Database (MSigDB) hallmark gene set collection, Cell Syst, 1 (2015) 417-425.
A. Welsh, S. A. Eschrich, A. E. Berglund, D. A. Fenstermacher, Iterative rank-order normalization of gene expression microarray data, BMC Bioinformatics, 14 (2013) 153.
A. J. Lee, A. A. Ashkar, The Dual Nature of Type I and Type II Interferons, Front Immunol, 9 (2018) 2061.
A. M. Lesokhin, M. K. Callahan, M. A. Postow, J. D. Wolchok, On being less tolerant: enhanced cancer immunosurveillance enabled by targeting checkpoints and agonists of T cell activation, Science translational medicine, 7 (2015) 280sr281.
B. L. Bunch, K. N. Kodumudi, E. Scott, J. Morse, A. M. Weber, A. E. Berglund, S. Pilon-Thomas, J. Markowitz, Anti-tumor efficacy of plasmid encoding emm55 in a murine melanoma model, Cancer immunology, immunotherapy: CII, (2020). June 18. doi: 10.1007/s00262-020-02634-4, online ahcad of print.
B. L. Mundy-Bosse, G. S. Young, T. Bauer, E. Binkley, M. Bloomston, M. A. Bill, T. Bekaii-Saab, W. E. Carson, 3rd, G. B. Lesinski, Distinct myeloid suppressor cell subsets correlate with plasma IL-6 and IL-10 and reduced interferon-alpha signaling in CD4(+) T cells from patients with GI malignancy, Cancer immunology, immunotherapy: CII, 60 (2011) 1269-1279.
Bassett, V. Gopalakrishnan, K. Wani, M. P. De Macedo, J. L. Austin-Breneman, H. Jiang, Q. Chang, S. M. Reddy, W. S. Chen, M. T. Tetzlaff, R. J. Broaddus, M. A. Davies, J. E. Gershenwald, L. Haydu, A. J. Lazar, S. P. Patel, P. Hwu, W. J. Hwu, A. Diab, I. C. Glitza, S. E. Woodman, L. M. Vence, Wistuba, II, R. N. Amaria, L. N. Kwong, V. Prieto, R. E. Davis, W. Ma, W. W. Overwijk, A. H. Sharpe, J. Hu, P. A. Futreal, J. Blando, P. Sharma, J. P. Allison, L. Chin, J. A. Wargo, Analysis of Immune Signatures in Longitudinal Tumor Samples Yields Insight into Biomarkers of Response and Mechanisms of Resistance to Immune Checkpoint Blockade, Cancer Discov, 6 (2016) 827-837.
Brinkmann, V., et al., Interferon alpha increases the frequency of interferon gamma-producing human CD4⁺ T cells. J Exp Med, 1993. 178(5): p. 1655-63.
Cook, J. and J. A. Zitelli, Treating patients with melanoma with interferon. Arch Dermatol, 1997. 133(3): p. 387-9.
D. I. Gabrilovich, S. Ostrand-Rosenberg, V. Bronte, Coordinated regulation of myeloid cells by tumours, Nature Reviews Immunology, 12 (2012) 253-268.
Davar, D. and J. M. Kirkwood, Adjuvant Therapy of Melanoma. Cancer Treat Res, 2016. 167: p. 181-208.
Di Trolio, R., et al., The use of interferon in melanoma patients: a systematic review. Cytokine Growth Factor Rev, 2015. 26(2): p. 203-12.
Diamond, M. S., et al., Type I interferon is selectively required by dendritic cells for immune rejection of tumors. J Exp Med, 2011. 208(10): p. 1989-2003.
Erkes, W. Cai, I. M. Sanchez, T. J. Purwin, C. Rogers, C. O. Field, A. C. Berger, E. J. Hartsough, U. Rodeck, E. S. Alnemri, A. E. Aplin, Mutant BRAF and MEK Inhibitors Regulate the Tumor Immune Microenvironment via Pyroptosis, Cancer Discov, 10 (2020) 254-269.
G. B. Lesinski, M. Anghelina, J. Zimmerer, T. Bakalakos, B. Badgwell, R. Parihar, Y. Hu, B. Becknell, G. Abood, A. R. Chaudhury, C. Magro, J. Durbin, W. E. Carson, 3rd, The antitumor effects of IFN-alpha are abrogated in a STAT1-deficient mouse, The Journal of clinical investigation, 112 (2003) 170-180.
G. B. Lesinski, S. V. Kondadasula, T. Crespin, L. Shen, K. Kendra, M. Walker, W. E. Carson, 3rd, Multiparametric flow cytometric analysis of inter-patient variation in STAT1 phosphorylation following interferon Alfa immunotherapy, Journal of the National Cancer Institute, 96 (2004) 1331-1342.
G. K. Pennock, L. Q. Chow, The Evolving Role of Immune Checkpoint Inhibitors in Cancer Treatment, The oncologist, 20 (2015) 812-822.
Gabrilovich, D., Mechanisms and functional significance of tumour-induced dendritic-cell defects. Nat Rev Immunol, 2004. 4(12): p. 941-52.
Gabrilovich, Mechanisms and functional significance of tumour-induced dendritic-cell
Gajewski, T. F., M. B. Fuertes, and S. R. Woo, Innate immune sensing of cancer: clues from an identified role for type I IFNs. Cancer Immunol Immunother, 2012. 61(8): p. 1343-7.
Garg, S. K., et al., Multi-Dimensional Flow Cytometry Analyses Reveal a Dichotomous Role for Nitric Oxide in Melanoma Patients Receiving Immunotherapy. Front Immunol, 2020. 11: p. 164.
Garg, S. K., et al., Multi-Omics and Informatics Analysis of FFPE Tissues Derived from Melanoma Patients with Long/Short Responses to Anti-PD1 Therapy Reveals Pathways of Response. Cancers (Basel), 2020. 12(12).
Gebhardt, A. Sevko, H. Jiang, R. Lichtenberger, M. Reith, K. Tarnanidis, T. Holland Letz, L. Umansky, P. Beckhove, A. Sucker, D. Schadendorf, J. Utikal, V. Umansky, Myeloid Cells and Related Chronic Inflammatory Factors as Novel Predictive Markers in Melanoma Treatment with Ipilimumab, Clinical cancer research: an official journal of the American Association for Cancer Research, 21 (2015) 5453-5459.
Grasso, C. S., et al., Conserved Interferon-gamma Signaling Drives Clinical Response to Immune Checkpoint Blockade Therapy in Melanoma. Cancer Cell, 2021. 39(1): p. 122.
Grossmann, K. F., et al., Adjuvant Pembrolizumab versus IFNalpha2b or Ipilimumab in Resected High-Risk Melanoma. Cancer Discov, 2022. 12(3): p. 644-653.
Homet Moreno, G. Parisi, L. Robert, A. Ribas, Anti-PD-1 therapy in melanoma, Seminars in oncology, 42 (2015) 466-473.
J. Jarboe, A. Gupta, W. Saif, Therapeutic human monoclonal antibodies against cancer, Methods Mol Biol, 1060 (2014) 61-77.
J. Larkin, C. D. Lao, W. J. Urba, D. F. McDermott, C. Horak, J. Jiang, J. D. Wolchok, Efficacy and Safety of Nivolumab in Patients With BRAF V600 Mutant and BRAF Wild-Type Advanced Melanoma: A Pooled Analysis of 4 Clinical Trials, JAMA oncology, 1 (2015) 433-440.
J. Markowitz, J. Wang, Z. Vangundy, J. You, V. Yildiz, L. Yu, I. P. Foote, O. E. Branson, A. R. Stiff, T. R. Brooks, B. Biesiadecki, T. Olencki, S. Tridandapani, M. A. Freitas, T. Papenfuss, M. A. Phelps, W. E. Carson, Nitric oxide mediated inhibition of antigen presentation from DCs to CD4(+) T cells in cancer and measurement of STAT1 nitration, Sci Rep, 7 (2017) 15424.
J. Neefjes, M. L. Jongsma, P. Paul, O. Bakke, Towards a systems understanding of MHC class I and MHC class II antigen presentation, Nature reviews. Immunology, 11 (2011) 823-836.
J. Oh, A. L. Byrd, M. Park, N. C. S. Program, H. H. Kong, J. A. Segre, Temporal Stability of the Human Skin Microbiome, Cell, 165 (2016) 854-866.
J. Wang, C. J. Perry, K. Meeth, D. Thakral, W. Damsky, G. Micevic, S. Kaech, K. Blenman, M. Bosenberg, UV-induced somatic mutations elicit a functional T cell response in the YUMMER1.7 mouse melanoma model, Pigment Cell Melanoma Res, 30 (2017) 428-435.
J. Wasserman, L. Diese, Z. VanGundy, C. London, W. E. Carson, T. L. Papenfuss, Suppression of canine myeloid cells by soluble factors from cultured canine tumor cells, Veterinary immunology and immunopathology, 145 (2012) 420-430.
J. Weber, G. Gibney, R. R. Kudchadkar, B. Yu, P. Cheng, A. J. Martinez, J. Kroeger, A.
J. M. Zaretsky, A. Garcia-Diaz, D. S. Shin, H. Escuin-Ordinas, W. Hugo, S. Hu-Lieskovan, D. Y. Torrejon, G. Abril-Rodriguez, S. Sandoval, L. Barthly, J. Saco, B. Homet Moreno, R. Mezzadra, B. Chmielowski, K. Ruchalski, I. P. Shintaku, P. J. Sanchez, C. Puig-Saus, G. Cherry, E. Seja, X. Kong, J. Pang, B. Berent-Maoz, B. Comin-Anduix, T. G. Graeber, P. C. Tumeh, T. N. Schumacher, R. S. Lo, A. Ribas, Mutations Associated with Acquired Resistance to PD-1 Blockade in Melanoma, N Engl J Med, 375 (2016) 819-829.
Jacquelot, N., et al., Sustained Type I interferon signaling as a mechanism of resistance to PD-1 blockade. Cell Res, 2019. 29(10): p. 846-861.
Johnson, M. V. Estrada, R. Salgado, V. Sanchez, D. B. Doxie, S. R. Opalenik, A. E. Vilgelm, E. Feld, A. S. Johnson, A. R. Greenplate, M. E. Sanders, C. M. Lovly, D. T. Frederick, M. C. Kelley, A. Richmond, J. M. Irish, Y. Shyr, R. J. Sullivan, I. Puzanov, J. A. Sosman, J. M. Balko, Melanoma-specific MHC-II expression represents a tumour-autonomous phenotype and predicts response to anti-PD-1/PD-L1 therapy, Nat Commun, 7 (2016) 10582.
K. Taniguchi, M. Karin, NF-kappaB, inflammation, immunity and cancer: coming of age, Nature reviews. Immunology, 18 (2018) 309-324.
Kirkwood, J. M., et al., Immunomodulatory effects of high-dose and low-dose interferon alpha2b in patients with high-risk resected melanoma: the E2690 laboratory corollary of intergroup adjuvant trial E1690. Cancer, 2002. 95(5): p. 1101-12.
L. Chen, X. Han, Anti-PD-1/PD-L1 therapy of human cancer: past, present, and future, The Journal of clinical investigation, 125 (2015) 3384-3391.
L. Mundy-Bosse, G. B. Lesinski, A. C. Jaime-Ramirez, K. Benninger, M. Khan, P. Kuppusamy, K. Guenterberg, S. V. Kondadasula, A. R. Chaudhury, K. M. La Perle, M. Kreiner, G. Young, D. C. Guttridge, W. E. Carson, 3rd, Myeloid-derived suppressor cell inhibition of the IFN response in tumor-bearing mice, Cancer research, 71 (2011) 5101-5110.
Larkin, J., et al., Adjuvant nivolumab versus ipilimumab (CheckMate 238 trial): Reassessment of 4-year efficacy outcomes in patients with stage III melanoma per AJCC-8 staging criteria. Eur J Cancer, 2022. 173: p. 285-296.
Lesinski, G. B., et al., Multiparametric flow cytometric analysis of inter-patient variation in STAT1 phosphorylation following interferon Alfa immunotherapy. J Natl Cancer Inst, 2004. 96(17): p. 1331-42.
M. Di Napoli, F. Papa, NCX-4016 NicOx, Curr Opin Investig Drugs, 4 (2003) 1126-1139.
M. Montoya, G. Schiavoni, F. Mattei, I. Gresser, F. Belardelli, P. Borrow, D. F. Tough, Type I interferons produced by dendritic cells promote their phenotypic and functional activation, Blood, 99 (2002) 3263-3271.
M. A. Postow, M. K. Callahan, J. D. Wolchok, Immune Checkpoint Blockade in Cancer Therapy, Journal of clinical oncology: official journal of the American Society of Clinical Oncology, 33 (2015) 1974-1982.
M. S. Diamond, M. Kinder, H. Matsushita, M. Mashayekhi, G. P. Dunn, J. M. Archambault, H. Lee, C. D. Arthur, J. M. White, U. Kalinke, K. M. Murphy, R. D. Schreiber, Type I interferon is selectively required by dendritic cells for immune rejection of tumors, The Journal of experimental medicine, 208 (2011) 1989-2003.
Margulis, A. Sitaramayya, Rate of deactivation of nitric oxide-stimulated soluble guanylate cyclase: influence of nitric oxide scavengers and calcium, Biochemistry, 39 (2000) 1034-1039.
Markowitz, J., et al., Nitric oxide mediated inhibition of antigen presentation from DCs to CD4 (±) T cells in cancer and measurement of STAT1 nitration. Sci Rep, 2017. 7(1): p. 15424.
Mundy-Bosse, B. L., et al., Myeloid-derived suppressor cell inhibition of the IFN response in tumor-bearing mice. Cancer Res, 2011. 71(15): p. 5101-10.
P. Darvin, S. M. Toor, V. Sasidharan Nair, E. Elkord, Immune checkpoint inhibitors: recent progress and potential biomarkers, Exp Mol Med, 50 (2018) 165.
P. Minuz, M. Degan, S. Gaino, A. Meneguzzi, V. Zuliani, C. L. Santonastaso, P. D. Soldato, A. Lechi, NCX4016 (NO-Aspirin) has multiple inhibitory effects in LPS-stimulated human monocytes, Br J Pharmacol, 134 (2001) 905-911.
P. Muranski, A. Boni, P. A. Antony, L. Cassard, K. R. Irvine, A. Kaiser, C. M. Paulos, D. C. Palmer, C. E. Touloukian, K. Ptak, L. Gattinoni, C. Wrzesinski, C. S. Hinrichs, K. W. Kerstann, L. Feigenbaum, C. C. Chan, N. P. Restifo, Tumor-specific Th17-polarized cells eradicate large established melanoma, Blood, 112 (2008) 362-373.
P. L. Chen, W. Roh, A. Reuben, Z. A. Cooper, C. N. Spencer, P. A. Prieto, J. P. Miller, R. L.
P. S. Ljubuncic, M. Bar-Shai, A. Z. Reznick, The Role of Reactive Nitrogen Species (RNS) in theActivation of Nuclear Factor Kappa B (NFkB) and its Implications for Biological Symptoms, in: G. Valacchi, P. A. Davis (Eds.) Oxidants in Biology, Springer, Place Published, 2008, pp. 67-109.
Palmer, K. J., et al., Interferon-alpha (IFN-alpha) stimulates anti-melanoma cytotoxic T lymphocyte (CTL) generation in mixed lymphocyte tumour cultures (MLTC). Clin Exp Immunol, 2000. 119(3): p. 412-8.
R. Radi, Protein tyrosine nitration: biochemical mechanisms and structural basis of functional effects, Acc Chem Res, 46 (2013) 550-559.
R. L. Siegel, K. D. Miller, A. Jemal, Cancer statistics, 2020, CA Cancer J Clin, 70 (2020) 7-30.
R. M. Bingisser, P. A. Tilbrook, P. G. Holt, U. R. Kees, Macrophage-derived nitric oxide regulates T cell activation via reversible disruption of the Jak3/STAT5 signaling pathway, Journal of immunology, 160 (1998) 5729-5734.
Radi, R., Protein tyrosine nitration: biochemical mechanisms and structural basis of functional effects. Acc Chem Res, 2013. 46(2): p. 550-9.
Reich, N.C., STAT dynamics. Cytokine & Growth Factor Reviews, 2007. 18(5-6): p. 511-518.
Richards, L. McCormick, V. Moberg, H. Cronin, X. Zhao, M. Schell, Y. A. Chen, Phase I/II study of metastatic melanoma patients treated with nivolumab who had progressed after ipilimumab, Cancer Immunol Res, (2016).
Robert, A. Ribas, J. Schachter, A. Arance, J. J. Grob, L. Mortier, A. Daud, M. S. Carlino, C. M. McNeil, M. Lotem, J. M. G. Larkin, P. Lorigan, B. Neyns, C. U. Blank, T. M. Petrella, O. Hamid, S. C. Su, C. Krepler, N. Ibrahim, G. V. Long, Pembrolizumab versus ipilimumab in advanced melanoma (KEYNOTE-006): post-hoc 5-year results from an open-label, multicentre, randomised, controlled, phase 3 study, Lancet Oncol, 20 (2019) 1239-1251.
S. Feng, X. Cheng, L. Zhang, X. Lu, S. Chaudhary, R. Teng, C. Frederickson, M. M. Champion, R. Zhao, L. Cheng, Y. Gong, H. Deng, X. Lu, Myeloid-derived suppressor cells inhibit T cell activation through nitrating LCK in mouse cancers, Proc Natl Acad Sci USA, 115 (2018) 10094-10099.
S. Hervas-Stubbs, J. L. Perez-Gracia, A. Rouzaut, M. F. Sanmamed, A. Le Bon, I. Melero, Direct effects of type I interferons on cells of the immune system, Clinical cancer research: an official journal of the American Association for Cancer Research, 17 (2011) 2619-2627.
S. Nagaraj, K. Gupta, V. Pisarev, L. Kinarsky, S. Sherman, L. Kang, D. L. Herber, J. Schneck, D. I. Gabrilovich, Altered recognition of antigen is a mechanism of CD8+ T cell tolerance in cancer, Nat Med, 13 (2007) 828-835.
S. Pilon-Thomas, K. N. Kodumudi, A. E. El-Kenawi, S. Russell, A. M. Weber, K. Luddy, M. Damaghi, J. W. Wojtkowiak, J. J. Mule, A. Ibrahim-Hashim, R. J. Gillies, Neutralization of Tumor Acidity Improves Antitumor Responses to Immunotherapy, Cancer research, 76 (2016) 1381-1390.
S. Pilon-Thomas, N. Nelson, N. Vohra, M. Jerald, L. Pendleton, K. Szekeres, T. Ghansah, Murine pancreatic adenocarcinoma dampens SHIP-1 expression and alters MDSC homeostasis and function, PLOS One, 6 (2011) e27729.
S. Shin, J. M. Zaretsky, H. Escuin-Ordinas, A. Garcia-Diaz, S. Hu-Lieskovan, A. Kalbasi, C. S. Grasso, W. Hugo, S. Sandoval, D. Y. Torrejon, N. Palaskas, G. A. Rodriguez, G. Parisi, A. Azhdam, B. Chmielowski, G. Cherry, E. Seja, B. Berent-Maoz, I. P. Shintaku, D. T. Le, D. M. Pardoll, L. A. Diaz, Jr., P. C. Tumch, T. G. Gracber, R. S. Lo, B. Comin-Anduix, A. Ribas, Primary Resistance to PD-1 Blockade Mediated by JAK1/2 Mutations, Cancer Discov, 7 (2017) 188-201.
S. Tyanova, T. Temu, J. Cox, The MaxQuant computational platform for mass spectrometry-based shotgun proteomics, Nat Protoc, 11 (2016) 2301-2319.
S. J. Rodig, D. Gusenleitner, D. G. Jackson, E. Gjini, A. Giobbie-Hurder, C. Jin, H. Chang, S. B. Lovitch, C. Horak, J. S. Weber, J. L. Weirather, J. D. Wolchok, M. A. Postow, A. C. Pavlick, J. Chesney, F. S. Hodi, MHC proteins confer differential sensitivity to CTLA-4 and PD-1 blockade in untreated metastatic melanoma, Science translational medicine, 10 (2018).
S. K. Garg, M. J. Ott, A. G. M. Mostofa, Z. Chen, Y. A. Chen, J. Kroeger, B. Cao, A. W. Mailloux, A. Agrawal, B. J. Schaible, A. Sarnaik, J. S. Weber, A. E. Berglund, J. J. Mule, J. Markowitz, Multi-Dimensional Flow Cytometry Analyses Reveal a Dichotomous Role for Nitric Oxide in Melanoma Patients Receiving Immunotherapy, Front Immunol, 11 (2020) 164.
S. W. Park, M. D. Huq, X. Hu, L. N. Wei, Tyrosine nitration on p65: a novel mechanism to rapidly inactivate nuclear factor-kappaB, Mol Cell Proteomics, 4 (2005) 300-309.
Sarnaik, A. A., et al., Extended dose ipilimumab with a peptide vaccine: immune correlates associated with clinical benefit in patients with resected high-risk stage IIIc/IV melanoma. Clin Cancer Res, 2011. 17(4): p. 896-906.
Simons, D. L., et al., Interferon signaling patterns in peripheral blood lymphocytes may predict clinical outcome after high-dose interferon therapy in melanoma patients. J Transl Med, 2011. 9: p. 52.
Suarez-Kelly, L. P., et al., A pilot study of interferon-alpha-2b dose reduction in the adjuvant therapy of high-risk melanoma. Cancer Immunol Immunother, 2019. 68(4): p. 619-629.
Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles, Proc Natl Acad Sci USA, 102 (2005) 15545-15550.
T. Ito, R. Amakawa, M. Inaba, S. Ikehara, K. Inaba, S. Fukuhara, Differential regulation of human blood dendritic cell subsets by IFNs, Journal of immunology, 166 (2001) 2961-2969.
T. Lu, R. Ramakrishnan, S. Altiok, J. I. Youn, P. Cheng, E. Celis, V. Pisarev, S. Sherman, M. B. Sporn, D. Gabrilovich, Tumor-infiltrating myeloid cells induce tumor cell resistance to cytotoxic T cells in mice, The Journal of clinical investigation, 121 (2011) 4015-4029.
T. F. Gajewski, M. B. Fuertes, S. R. Woo, Innate immune sensing of cancer: clues from an identified role for type I IFNs, Cancer immunology, immunotherapy: CII, 61 (2012) 1343-1347.
Tarhini, A. A., Adjuvant Therapy of Melanoma. Hematol Oncol Clin North Am, 2021. 35(1): p. 73-84.
Tarhini, A. A., et al., Phase III Study of Adjuvant Ipilimumab (3 or 10 mg/kg) Versus High-Dose Interferon Alfa-2b for Resected High-Risk Melanoma: North American Intergroup E1609. J Clin Oncol, 2020. 38(6): p. 567-575.
Tarhini, A. A., H. Gogas, and J. M. Kirkwood, IFN-alpha in the treatment of melanoma. J Immunol, 2012. 189(8): p. 3789-93.
Testori, A. A. E., S. Chiellino, and A. C. J. van Akkooi, Adjuvant Therapy for Melanoma: Past, Current, and Future Developments. Cancers (Basel), 2020. 12(7).
Tough, D. F., Type I interferon as a link between innate and adaptive immunity through dendritic cell stimulation. Leuk Lymphoma, 2004. 45(2): p. 257-64.
V. A. Zavala, A. M. Kalergis, New clinical advances in immunotherapy for the treatment of solid tumours, Immunology, 145 (2015) 182-201.
Weber, J., et al., Adjuvant Nivolumab versus Ipilimumab in Resected Stage III or IV Melanoma. N Engl J Med, 2017. 377(19): p. 1824-1835.
Y. Liang, H. Tang, J. Guo, X. Qiu, Z. Yang, Z. Ren, Z. Sun, Y. Bian, L. Xu, H. Xu, J. Shen, Y. Han, H. Dong, H. Peng, Y. X. Fu, Targeting IFNalpha to tumor by anti-PD-L1 creates feedforward antitumor responses to overcome checkpoint blockade resistance, Nat Commun, 9 (2018) 4586.
Yarlagadda, K., et al., The role of nitric oxide in melanoma. Biochim Biophys Acta Rev Cancer, 2017. 1868(2): p. 500-509.
Zimmerer, J. M., et al., IFN-alpha-2b-induced signal transduction and gene regulation in patient peripheral blood mononuclear cells is not enhanced by a dose increase from 5 to 10 megaunits/m2. Clin Cancer Res, 2008. 14(5): p. 1438-45.

What is claimed is:

1. A method of reducing immune checkpoint inhibitor resistance comprising administering a nitric oxide inhibitor to a subject being treated for a disease with an immune checkpoint inhibitor.

2. The method of reducing immune checkpoint inhibitor resistance of claim 1, wherein the nitric oxide inhibitor is administered no greater than 3 days after administration of the immune checkpoint inhibitor.

3. The method of reducing immune checkpoint inhibitor resistance of claim 2, wherein the nitric oxide inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

4. The method of reducing immune checkpoint inhibitor resistance of any of claims 1-3, wherein the immune checkpoint inhibitor comprises Nivolumab (BMS-936558 or MDX1106), CT-011, MK-3475, MDX-1105 (BMS-936559), MPDL3280A, or MSB0010718C, rHIgM12B7, Ipilimumab (MDX-010), Tremelimumab (CP-675,206), antibodies or small molecules that specifically bind IDO, B7-H3 (MGA271), B7-H4, TIM3, or LAG-3 (BMS-986016).

5. The method of reducing immune checkpoint inhibitor resistance of any of claims 1-4, wherein the nitric oxide inhibitor is administered prior to day 3 after administration of the immune checkpoint inhibitor.

6. A combination therapy comprising an immune checkpoint inhibitor and a nitric oxide inhibitor.

7. The combination therapy of claim 6, wherein the nitric oxide inhibitor comprises NCX-4016, L-NAME, N(G)-monomethyl L-arginine (L-NMMA), Aminoguanidine hydrochloride, S-Isopropylisothiourea hydrobromide, or (S)-Methylisothiourea sulfate.

8. The combination therapy of claim 6 or 7, wherein the immune checkpoint inhibitor comprises Nivolumab (BMS-936558 or MDX1106), CT-011, MK-3475, MDX-1105 (BMS-936559), MPDL3280A, or MSB0010718C, rHIgM12B7, Ipilimumab (MDX-010), Tremelimumab (CP-675,206), antibodies or small molecules that specifically bind IDO, B7-H3 (MGA271), B7-H4, TIM3, or LAG-3 (BMS-986016).

9. A method of treating a cancer in a subject comprising administering to the subject a nitric oxide inhibitor and an immune checkpoint inhibitor.

10. The method of treating a cancer of claim 9, wherein the nitric oxide inhibitor is administered no greater than 3 days after administration of the immune checkpoint inhibitor.

11. The method of treating a cancer of claim 9 or 10, wherein the nitric oxide inhibitor comprises NCX-4016, L-nitroarginine methyl ester (L-NAME), N(G)-monomethyl L-arginine (L-NMMA), Aminoguanidine hydrochloride, S-Isopropylisothiourea hydrobromide, or (S)-Methylisothiourea sulfate.

12. The method of treating a cancer of any of claims 9-11, wherein the immune checkpoint inhibitor comprises Nivolumab (BMS-936558 or MDX1106), CT-011, MK-3475, MDX-1105 (BMS-936559), MPDL3280A, or MSB0010718C, rHIgM12B7, Ipilimumab (MDX-010), Tremelimumab (CP-675,206), antibodies or small molecules that specifically bind IDO, B7-H3 (MGA271), B7-H4, TIM3, or LAG-3 (BMS-986016).

13. The method of treating a cancer of any of claims 9-12, wherein the nitric oxide inhibitor is administered prior to day 3 after administration of the immune checkpoint inhibitor.

14. The method of treating a cancer of any of claims 9-13, wherein the cancer comprises melanoma.

15. A method of detecting resistance of a disease or condition in a subject to an immune checkpoint inhibitor comprising:
a. obtaining a tissue sample from a subject that is receiving or that is likely to receive an immune checkpoint inhibitor for treatment of a disease or condition; and
b. measuring the level of nitration in the tissue sample; wherein an increase in nitration relative to a control indicates that the disease or condition is resistant to an immune checkpoint inhibitor; and
c. when a subject has a disease or condition that is resistant to an immune checkpoint inhibitor, administering to the subject a nitric oxide inhibitor.

16. The method of claim 15, wherein the disease or condition is a cancer.

17. The method of claim 15, wherein the disease or condition comprises immune checkpoint inhibitor toxicity.

18. The method of any of claims 15-17, wherein the immune checkpoint inhibitor comprises Nivolumab (BMS-936558 or MDX1106), CT-011, MK-3475, MDX-1105 (BMS-936559), MPDL3280A, or MSB0010718C, rHIgM12B7, Ipilimumab (MDX-010), Tremelimumab (CP-675,206), antibodies or small molecules that specifically bind IDO, B7-H3 (MGA271), B7-H4, TIM3, or LAG-3 (BMS-986016).

19. The method of any of claims 15-18, wherein the nitic oxide inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

20. The method of any of claims 15-19, wherein nitration is measured by one or more of mass spectroscopy, flow cytometry, immunohistochemical staining, Multi-Dimensional Phenotyping Analysis Tool in R (MPATR) clustering, and/or proteomics.

21. A method of improving progression free survival (PFS) in a subject with a cancer resistant to conventional cancer therapy, the method comprising:
a) obtaining biological sample from the subject,
b) performing a multi-parameter phenotyping tool in R (MPATR) assay on the biological sample to analyze the nitric oxide (NO) levels in the biological sample;
c) measuring the immune cell subtype producing NO; wherein the immune cell subtype are immune suppressive cells, or immune effector cells; wherein an increased level of NO in immune suppressive cells is an indication of cancer subject resistance to conventional cancer therapy; wherein NO in immune suppressive cells lead to shorter PFS,
d) administering a NO inhibitor in combination with an immune checkpoint inhibitor to subjects with a cancer resistant to conventional cancer therapy having increased levels of NO in immune suppressive cells.

22. The method of claim 21, wherein the cancer comprises carcinoma, melanoma, lymphoma, sarcoma, or leukemia.

23. The method of claim 21, wherein the immune suppressive cells are myeloid-derived suppressor cells (MDSC), regulatory T cells (Tregs), or tumor macrophages.

24. The method of claim 21, wherein the immune effector cells are CD8+ cytotoxic T cells, CD4+ helper T cells, DC cells, natural killer (NK) cells, NK T cells or B cells.

25. The method of claim 21, wherein the NO inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

26. The method of claim 21, wherein the conventional cancer therapy comprises immune checkpoint inhibitors comprising anti-PD-1, anti-PD-L1, and/or anti-CTLA-4; or interferon-based therapy.

27. The method of claim 21, wherein the biological sample comprises peripheral blood mononuclear cells (PBMC).

28. The method of claim 21, wherein the NO inhibitor is administered on day 2 and administration of immune checkpoint inhibitor therapy on day 0.

29. A multi-parameter phenotyping tool in R (MPATR) assay to identify nitration of proteins in an immune cell subtype, comprising:
a) obtaining biological sample from a subject with a cancer that is resistant to conventional cancer therapy,
b) measuring the immune cell subtype producing NO; wherein the immune cell subtype are immune suppressive cells or immune effector cells; and
c) measuring the nitration of proteins in the immune cells; wherein an increased level of nitration of proteins in immune suppressive cells is an indication that the subject needs a NO inhibitor to overcome resistance to conventional cancer therapy.

30. The assay of claim 29, wherein the nitration of proteins in an immune cell subtype is measured using mass spectrometry analysis.

31. The assay of claim 29, wherein the subtype of the immune cells is measured using flow cytometry panels consisting of a myeloid cell panel, a lymphoid cell panel, and a secondary panel; wherein the panels were stained with myeloid, or lymphoid antibodies.

32. The assay of claim 31, wherein the myeloid panel antibodies comprise DAF-FM, HLA-DR-PE-Cy7, CD33-APC, CD11b-BV421, CD14-BUV395, CD15-BV510, or CD11c-PE.

33. The assay of claim 31, wherein the lymphoid panel antibodies comprise DAF-FM, CD3-BUV395, CD8-BV510, CD11c-PE, CD56-BV421, CD4-AF700, CD19-PE-Dazzle, CD25-PE-Cy7, or CD127-APC.

34. The assay of claim 31, wherein the secondary panel antibodies comprise IFNγ, PD-L1, CTLA4, Arginase 1, FoxP3, TCR-ζ, or CD69.

35. The assay of any one of claims 29-34, wherein the biological sample comprises peripheral blood mononuclear cells (PBMC).

36. The assay of any one of claims 29-35, wherein the biological sample comprises immune suppressor cells and immune effector cells, wherein the immune suppressor cells are myeloid derived suppressor cells (MDSC), Tregs, and/or tumor macrophages; and wherein the immune effector cells are CD8+ cytotoxic T cells, CD4+ helper T cells, NK T cells, DC subsets, natural killer (NK) cells, and/or B cells.

37. The assay of any one of claims 29-36, wherein the cancer comprises carcinoma, melanoma, lymphoma, sarcoma, or leukemia.

38. The assay of any one of claims 29-37, wherein the conventional cancer therapy comprises immune checkpoint inhibitors comprising anti-PD-1, anti-PD-L1, and/or anti-CTLA-4; or interferon-based therapy.

39. The assay of claim 29 or 30, wherein the proteins identified for nitration comprises STAT1, Actin, NF-kB, STAT3, PUS7, FAK1, or HDAC1.

40. A method of assessing the susceptibility of a cancer in a subject to an immune checkpoint inhibitor therapy comprising:
a) obtaining a biological sample from the subject;
b) detecting level of NO in the biological sample using flow cytometry;
c) measuring immune cell subtype and NO level in the sample; wherein the immune cell subtypes are immune suppressive cells, or immune effector cells;
d) analyzing the nitration of proteins associated with antigen presentation in the sample using LC-MS/MS;
wherein increased NO level in immune suppressive cells and/or increased level of nitration of proteins in immune suppressive cells indicate poor immune checkpoint inhibitor therapy response in the subject; and wherein an increased level of NO level in immune effector cells indicates prolonged progression free survival (PFS); and
e) administering NO inhibitor with the immune checkpoint inhibitor if NO is higher in immune suppressive cells.

41. The method of claim 40, wherein the wherein the NO inhibitor is administered on day 2 and administration of immune checkpoint inhibitor therapy on day 0.

42. The method of claim 40, wherein the cancer comprises carcinoma, melanoma, lymphoma, sarcoma, or leukemia.

43. The method of claim 40, wherein the immune suppressive cells are myeloid-derived suppressor cells (MDSC), regulatory T cells (Tregs), or tumor macrophages.

44. The method of claim 40, wherein the immune effector cells are CD8+ cytotoxic T cells, CD4+ helper T cells, DC subsets, natural killer (NK) cells, NK T cells or B cells.

45. The method of claim 40, wherein the NO inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

46. The method of claim 40, wherein the conventional cancer therapy comprises immune checkpoint inhibitors comprising anti-PD-1, anti-PD-L1, and/or anti-CTLA-4; or interferon-based therapy.

47. The method of claim 40, wherein the biological sample comprises peripheral blood mononuclear cells (PBMC).

48. A method of treating an unresectable stage III/IV melanoma in a subject resistant to immune checkpoint inhibitor therapy, comprising:
a) measuring chronic NO levels after one year in a biological sample of the subject using a multi-parameter phenotyping tool in R (MPATR) assay; wherein increased NO levels indicate shorter PFS and poor response to immune checkpoint inhibitor therapy alone; and
b) administering combination of NO inhibitor and immune checkpoint inhibitor to the subject if NO level is increased in immune suppressive cells.

49. The method of claim 48, wherein the NO inhibitor is administered on day 2 after the second dose of immune checkpoint inhibitor therapy.

50. The method of claim 48, wherein the immune suppressive cells are myeloid-derived suppressor cells (MDSC), regulatory T cells (Tregs), or tumor macrophages.

51. The method of claim 48, wherein the NO inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

52. The method of claim 48, wherein the immune checkpoint inhibitor therapy comprises anti-PD-1, anti-PD-L1, and/or anti-CTLA-4.

53. The method of claim 48, wherein the biological sample comprises peripheral blood mononuclear cells (PBMC).

54. A method of inhibiting epithelial to mesenchymal transition (EMT) in melanoma patients by administering combination of immune checkpoint inhibitors and NO inhibitors.

55. The method of claim 54, wherein the NO inhibitor comprises NCX-4016, L-nitroarginine methyl ester (L-NAME), N(G)-monomethyl L-arginine (L-NMMA), NOSH-Aspirin, Aminoguanidine hydrochloride, S-Isopropylisothiourea hydrobromide, or (S)-Methylisothiourea sulfate.

56. The method of claim 54, wherein the immune checkpoint inhibitor therapy comprises anti-PD-1, anti-PD-L1, and/or anti-CTLA-4.

57. A composite model to determine a progression free survival (PFS) of a subject with a cancer, wherein the composite model comprises:
a) obtaining a biological sample from the subject;
b) measuring immune cell subtype; wherein the immune cell subtype are immune suppressive cells, or immune effector cells;
c) measuring the nitration of proteins in the sample; wherein increased nitration of proteins in the immune suppressive cells indicate shorter PFS of the subject; and
d) determining the subject's response to immune checkpoint inhibitor therapy;
wherein an increased nitration of proteins in the immune suppressive cells is an indication of poor response to immune checkpoint inhibitor therapy.

58. The method of claim 57, wherein the biological sample comprises peripheral blood mononuclear cells (PBMC).
